(19) United States
(12) Patent Application Publication
(10) Pub. No.: US 2026/0253449 A1
(43) Pub. Date: Aug. 27, 2026

(54) SYSTEM

(71) Applicant: SoftBank Group Corp., Tokyo (JP)

(72) Inventor: Takashi SATAKE, Tokyo (JP)

(73) Assignee: SoftBank Group Corp., Tokyo (JP)

(21) Appl. No.: 19/537,720

(22) Filed: Feb. 12, 2026

(30) Foreign Application Priority Data
Feb. 21, 2025 (JP) 2025-027095

(51) Int. Cl. G06V 40/20; G06V 10/75; G06V 10/764; G06V 10/82

(52) U.S. Cl. CPC G06V 40/23; G06V 10/751; G06V 10/764; G06V 10/82

(57) ABSTRACT

The system according to the embodiment comprises a recording unit, a comparison unit, an analysis unit, an evaluation unit, and a proposal unit. The recording unit is configured to record movements in a healthy state. The comparison unit is configured to record daily movements. The analysis unit is configured to analyze data recorded by the recording unit and the comparison unit. The evaluation unit is configured to evaluate risks based on data analyzed by the analysis unit. The proposal unit is configured to propose countermeasures based on risks evaluated by the evaluation unit.

### CROSS-REFERENCE TO RELATED APPLICATION

The present application claims priority to and incorporates by reference the entire contents of Japanese Patent Application No. 2025-027095 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to efficiently detect fluctuations in daily movements of elderly people and evaluate potential risks.

### SUMMARY OF THE INVENTION

The system according to the embodiment comprises a recording unit, a comparison unit, an analysis unit, an evaluation unit, and a proposal unit. The recording unit is configured to record movements in a healthy state. The comparison unit is configured to record daily movements. The analysis unit is configured to analyze data recorded by the recording unit and the comparison unit. The evaluation unit is configured to evaluate risks based on data analyzed by the analysis unit. The proposal unit is configured to propose countermeasures based on risks evaluated by the evaluation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi®, or Bluetooth®, among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

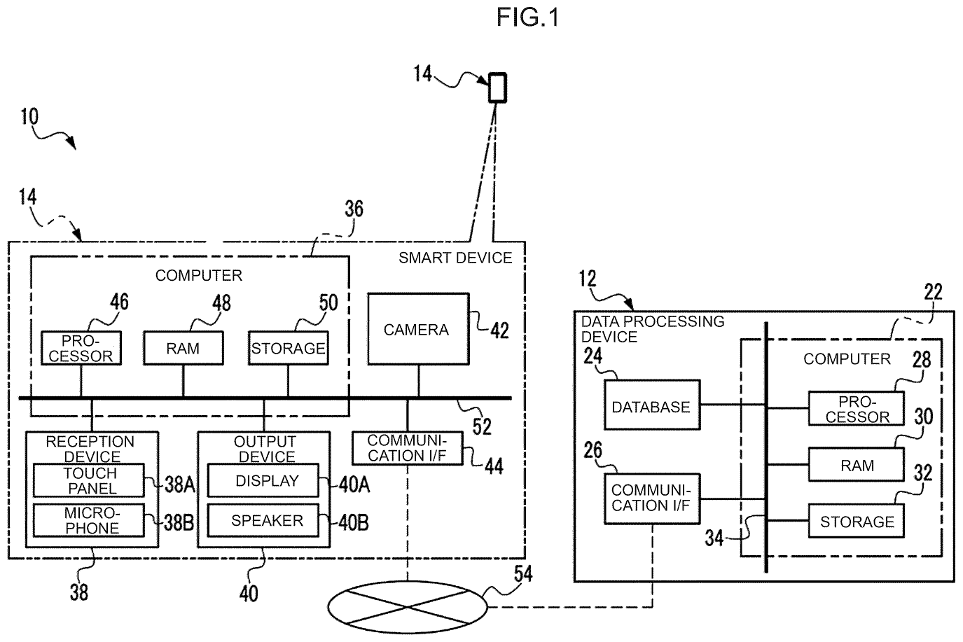

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

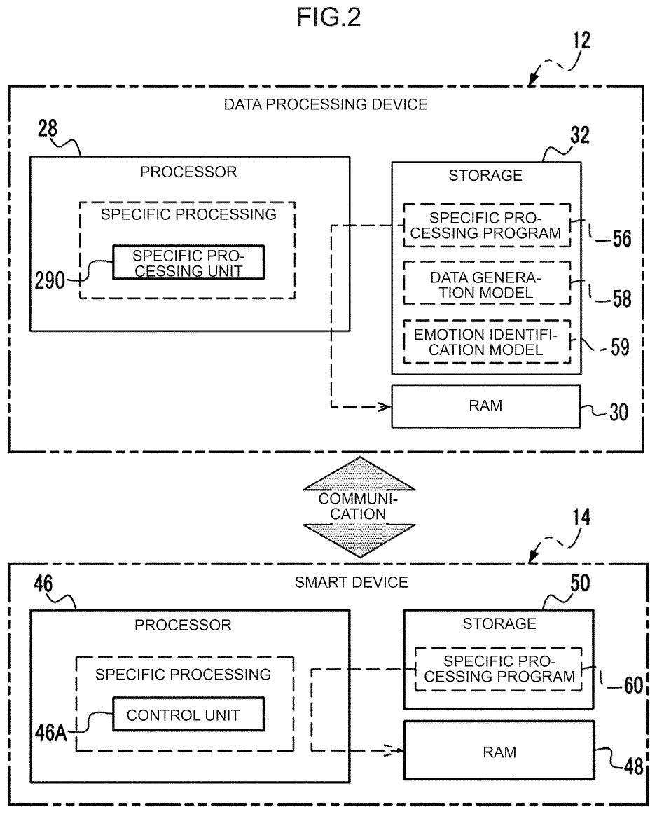

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The camera system according to the embodiment of the present invention is a system that inputs movements in a healthy state of elderly persons into AI and compares them with daily movements in their lives. This camera system captures movements in a healthy state of elderly persons using a camera and inputs the data into AI. Next, it captures daily movements using a camera, and the AI compares these data. If fluctuations occur in daily movements, such as walking style or eating habits, the generative AI analyzes the differences in movement and issues a warning in light of potential risks. For example, if walking speed decreases or the amount of food intake decreases, the generative AI detects this and evaluates the risk. If a high risk is determined, preventive healthcare or supplement recommendations are made, and countermeasures are proposed to the user. This enables elderly persons to easily take countermeasures suited to their symptoms. For example, the movements in a healthy state of elderly persons are captured by a camera. For example, posture and speed during walking, and eating habits are recorded in detail. This data is input into AI and stored as reference data. Next, daily movements are captured by a camera. For example, daily walking and eating conditions are recorded. This data is input into AI and compared with the data in a healthy state. The AI compares the data in a healthy state with daily data and detects fluctuations in movement. For example, if walking speed decreases or the amount of food intake decreases, the AI detects this. The generative AI analyzes these fluctuations and evaluates potential risks. If a high risk is determined, the generative AI recommends preventive healthcare or supplements. For example, if walking speed decreases, exercise therapy is proposed, and if the amount of food intake decreases, nutritional supplements are proposed. This enables elderly persons to easily take countermeasures suited to their symptoms. With this system, the health status of elderly persons can be monitored daily, and risks can be detected early. For example, by detecting fluctuations in daily life such as decreased walking speed or reduced food intake and proposing appropriate countermeasures, the system can contribute to maintaining health. Thus, the camera system can monitor the health status of elderly persons daily, detect risks early, and propose appropriate countermeasures. Specifically, this camera system is equipped with multiple high-resolution cameras and an image processing unit, and acquires motion data in healthy and daily states as time-series image tensors (e.g., number of frames×height×width×RGB channels, such as 60×224×224×3). The system performs noise removal, normalization, and pose estimation (e.g., skeleton extraction algorithms such as OpenPose) on these image tensors in a preprocessing unit, and inputs the obtained feature vectors (e.g., time-series arrays of joint coordinates, 60×18×2 dimensions) into a generative neural network (e.g., Transformer encoder-decoder structure with self-attention mechanism). The generative neural network encodes the reference motion vector in a healthy state and the daily motion vector, respectively, and extracts a difference vector. Examples of input to the AI include (1) sequences of skeletal coordinates during walking (e.g., 18 joints×2D coordinates for 60 frames), (2) motion vectors of hands, mouth, and tableware during eating (e.g., coordinates of major parts for 30 frames), and (3) image features for estimating food intake (e.g., pixel reduction in the area inside tableware). Outputs from the AI include (1) motion fluctuation score (e.g., 0.85 =large fluctuation), (2) risk label (e.g., “decreased walking,” “reduced food intake”), and (3) risk probability distribution (e.g., risk of decreased walking 0.72, risk of reduced food intake 0.65). The system inputs these output values into a threshold determination unit, generates a warning signal when the threshold is exceeded, and further transmits the risk type, importance, and recommended countermeasure category to the proposal unit. The proposal unit uses a pre-trained countermeasure generation model for each risk type (e.g., LLM-based proposal generator) to generate personalized countermeasure plans (e.g., “recommend light walking three times a week,” “recommend intake of protein supplements”) considering the user's health status, past countermeasure history, and preference information. This series of processing, unlike conventional human observation, recording, and judgment, performs automatic comparison, analysis, risk estimation, and countermeasure generation on image tensors and high-dimensional feature spaces at high speed and high accuracy, resulting in essential improvements in computer technology (e.g., improved monitoring accuracy, increased early risk detection rate, efficient data management, reduced user burden). Furthermore, by linking multiple AI models (e.g., CNN for pose estimation, Transformer for motion fluctuation detection, LLM for countermeasure generation), the system achieves both specialization at each processing stage and overall optimization, greatly improving the reliability and scalability of health monitoring. Application fields include elderly care facilities, home health management, rehabilitation support, and remote medical monitoring, and the system demonstrates significant technical effects especially in sites where labor shortages and limitations in observation accuracy are issues.

The camera system according to the embodiment comprises a recording unit, a comparison unit, an analysis unit, an evaluation unit, and a proposal unit. The recording unit records movements in a healthy state. For example, the recording unit can record posture or speed during walking, or eating habits. The recording unit, for example, captures posture during walking with a camera, and the AI analyzes the data. The recording unit can also capture eating habits with a camera and have the AI analyze the data. The comparison unit records daily movements. For example, the comparison unit can record daily walking or eating conditions. The comparison unit, for example, captures daily walking with a camera, and the AI analyzes the data. The comparison unit can also capture daily eating conditions with a camera and have the AI analyze the data. The analysis unit analyzes data recorded by the recording unit and the comparison unit. For example, the analysis unit compares data in a healthy state with daily data and detects fluctuations in movement. The analysis unit, for example, compares walking speed in a healthy state with daily walking speed and detects fluctuations. The analysis unit can also compare food intake in a healthy state with daily food intake and detect fluctuations. The evaluation unit evaluates risks based on data analyzed by the analysis unit. For example, the evaluation unit analyzes fluctuations in movement and evaluates potential risks. The evaluation unit, for example, evaluates decreased walking speed as a risk. The evaluation unit can also evaluate reduced food intake as a risk. The proposal unit proposes countermeasures based on risks evaluated by the evaluation unit. For example, if a high risk is determined, the proposal unit recommends preventive healthcare or supplements. The proposal unit, for example, proposes exercise therapy when walking speed decreases. The proposal unit can also propose nutritional supplements when food intake decreases. Thus, the camera system according to the embodiment can monitor the health status of elderly persons daily, detect risks early, and propose appropriate countermeasures. Specifically, the camera system is equipped with multiple high-resolution cameras and an image processing unit as the recording unit, and acquires motion data in a healthy state as time-series image tensors (e.g., 60 frames×224×224×3). The recording unit performs noise removal, normalization, and pose estimation (e.g., skeleton extraction algorithm) on the image tensors in a preprocessing unit, and inputs the obtained feature vectors (e.g., time-series arrays of joint coordinates, 60×18×2 dimensions) into a generative neural network (e.g., Transformer encoder-decoder structure). The comparison unit similarly acquires daily motion data and performs the same preprocessing and feature extraction as for the healthy state data. The analysis unit compares feature vectors in a healthy state and daily feature vectors and extracts difference vectors. Examples of input to the AI include sequences of skeletal coordinates during walking (60 frames of 18 joints×2D coordinates), motion vectors of hands, mouth, and tableware during eating (30 frames of major part coordinates), and image features for estimating food intake (pixel reduction in the area inside tableware). Outputs from the AI include motion fluctuation score (e.g., 0.85=large fluctuation), risk label (e.g., “decreased walking,” “reduced food intake”), and risk probability distribution (e.g., risk of decreased walking 0.72, risk of reduced food intake 0.65). The evaluation unit inputs these output values into a threshold determination unit, generates a warning signal when the threshold is exceeded, and transmits the risk type, importance, and recommended countermeasure category to the proposal unit. The proposal unit uses a pre-trained countermeasure generation model for each risk type (e.g., large language model-based proposal generator) to generate personalized countermeasure plans (e.g., “recommend light walking three times a week,” “recommend intake of protein supplements”) considering the user's health status, past countermeasure history, and preference information. This series of processing, unlike conventional human observation, recording, and judgment, performs automatic comparison, analysis, risk estimation, and countermeasure generation on image tensors and high-dimensional feature spaces at high speed and high accuracy, resulting in essential improvements in computer technology (e.g., improved monitoring accuracy, increased early risk detection rate, efficient data management, reduced user burden). Furthermore, by linking multiple AI models such as convolutional neural networks for pose estimation, Transformers for motion fluctuation detection, and large language models for countermeasure generation, the system achieves both specialization at each processing stage and overall optimization, greatly improving the reliability and scalability of health monitoring. Application fields include elderly care facilities, home health management, rehabilitation support, and remote medical monitoring, and the system demonstrates significant technical effects especially in sites where labor shortages and limitations in observation accuracy are issues.

The recording unit can record posture or speed during walking, or eating habits. For example, the recording unit captures posture during walking with a camera, and the AI analyzes the data. For example, the recording unit can record the degree of straightening of the back and the way the feet are moved. The recording unit can also capture walking speed with a camera and have the AI analyze the data. For example, the recording unit can measure walking speed and record movement speed. Furthermore, the recording unit can capture eating habits with a camera and have the AI analyze the data. For example, the recording unit can record the content, frequency, and amount of food intake. Thus, by recording detailed movements in a healthy state, the recording unit enables comparison with daily movements. Some or all of the above-described processing in the recording unit may be performed using AI or may be performed without using AI. For example, the recording unit can input data captured by a camera into a generative AI, and the generative AI can analyze the data. Specifically, the recording unit is equipped with multiple high-resolution cameras and an image processing unit, and acquires user movements during walking and eating as continuous image tensors (e.g., 60 frames×224×224×3). The recording unit performs noise removal, normalization, and pose estimation (e.g., skeleton extraction algorithm) on these image tensors in a preprocessing unit, and inputs the obtained feature vectors (e.g., time-series arrays of joint coordinates, 60×18×2 dimensions) into a generative neural network (e.g., Transformer encoder-decoder structure with self-attention mechanism). Examples of data input to the AI by the recording unit include (1) sequences of skeletal coordinates during walking (60 frames of 18 joints×2D coordinates), (2) motion vectors of hands, mouth, and tableware during eating (30 frames of major part coordinates), and (3) image features for estimating food intake (pixel reduction in the area inside tableware). Outputs received from the AI by the recording unit include (1) motion feature extraction vectors, (2) motion labels (e.g., “upright walking,” “stooped walking,” “dragging right foot”), (3) eating motion classification labels (e.g., “regular eating,” “interrupted eating”), and (4) estimated food intake values (e.g., intake amount 120 g). The recording unit structures and stores these output values in a recording database and provides data to subsequent comparison and analysis units. These processes, unlike conventional human observation and recording, perform automatic extraction, classification, and quantification on image tensors and high-dimensional feature spaces at high speed and high accuracy, resulting in essential improvements in computer technology such as improved recording accuracy, ensured objectivity of data, and reduced user burden through automation of recording tasks. Furthermore, by linking multiple AI models such as convolutional neural networks for pose estimation, Transformers for motion classification, and regression models for food intake estimation, the recording unit achieves both optimal feature extraction and improved recording accuracy for each recording target. Application fields include elderly care facilities, home health management, rehabilitation support, and remote medical monitoring, and the system demonstrates significant technical effects especially in sites where labor shortages and limitations in observation accuracy are issues.

The comparison unit can record daily walking or eating conditions. For example, the comparison unit captures daily walking with a camera, and the AI analyzes the data. For example, the comparison unit can record walking distance, walking time, and walking frequency. The comparison unit can also capture daily eating conditions with a camera and have the AI analyze the data. For example, the comparison unit can record the content, time, and frequency of meals. Thus, by recording daily movements, the comparison unit enables comparison with movements in a healthy state. Some or all of the above-described processing in the comparison unit may be performed using AI or may be performed without using AI. For example, the comparison unit can input data captured by a camera into a generative AI, and the generative AI can analyze the data. Specifically, the comparison unit, like the recording unit, uses multiple high-resolution cameras and image processing units to acquire daily walking and eating conditions as continuous image tensors (e.g., 60 frames×224×224×3). The comparison unit performs noise removal, normalization, and pose estimation (e.g., skeleton extraction algorithm) on these image tensors in a preprocessing unit, and inputs the obtained feature vectors (e.g., time-series arrays of joint coordinates, 60×18×2 dimensions) into a generative neural network (e.g., Transformer encoder-decoder structure). Examples of data input to the AI by the comparison unit include (1) sequences of skeletal coordinates during daily walking, (2) motion vectors of hands, mouth, and tableware during eating, and (3) image features for estimating food intake. Outputs received from the AI by the comparison unit include (1) daily motion feature extraction vectors, (2) motion labels (e.g., “stable walking,” “unstable walking”), (3) eating motion classification labels (e.g., “regular eating,” “interrupted eating”), and (4) estimated food intake values (e.g., intake amount 100 g). The comparison unit stores these output values in a recording database with the same structure as the healthy state data acquired by the recording unit, enabling automatic comparison by the subsequent analysis unit. These processes, unlike conventional human observation and recording, perform automatic extraction, classification, and quantification on image tensors and high-dimensional feature spaces at high speed and high accuracy, resulting in essential improvements in computer technology such as improved daily fluctuation detection accuracy, ensured objectivity of data, and reduced user burden through automation of recording tasks. Furthermore, by linking multiple AI models such as convolutional neural networks for pose estimation, Transformers for motion classification, and regression models for food intake estimation, the comparison unit achieves both optimal feature extraction and improved recording accuracy for each recording target. Application fields include elderly care facilities, home health management, rehabilitation support, and remote medical monitoring, and the system demonstrates significant technical effects especially in sites where labor shortages and limitations in observation accuracy are issues.

The analysis unit can compare data in a healthy state with daily data and detect fluctuations in movement. For example, the analysis unit compares data in a healthy state with daily data and detects fluctuations in movement. For example, the analysis unit can detect changes in walking speed or posture. The analysis unit can also detect changes in food intake. Thus, by detecting fluctuations in movement, the analysis unit can evaluate potential risks. Some or all of the above-described processing in the analysis unit may be performed using generative AI or may be performed without using generative AI. For example, the analysis unit can input data in a healthy state and daily data into a generative AI, and the generative AI can analyze the data. Specifically, the analysis unit inputs pairs of feature vectors in a healthy state and daily feature vectors (e.g., 60×18×2 skeletal coordinate sequences or eating motion vectors) obtained from the recording unit and the comparison unit into a generative neural network (e.g., Transformer encoder-decoder structure with self-attention mechanism) for encoding. The analysis unit calculates the difference vector between the healthy state vector and the daily vector in a high-dimensional space and outputs a motion fluctuation score (e.g., scalar value based on cosine similarity or Euclidean distance, 0.0-1.0). Examples of data input to the AI by the analysis unit include (1) pairs of skeletal coordinate sequences during walking in a healthy state and daily walking, (2) pairs of eating motion vectors in a healthy state and daily eating, and (3) pairs of estimated food intake values in a healthy state and daily eating. Outputs received from the AI by the analysis unit include (1) motion fluctuation score (e.g., 0.85=large fluctuation), (2) fluctuation type label (e.g., “decreased walking speed,” “posture collapse,” “reduced food intake”), and (3) fluctuation probability distribution (e.g., risk of decreased walking speed 0.72, risk of reduced food intake 0.65). The analysis unit transmits these output values to the subsequent evaluation unit for use as basis data for threshold determination and risk evaluation. These processes, unlike conventional human observation, recording, and judgment, perform automatic comparison and fluctuation detection on image tensors and high-dimensional feature spaces at high speed and high accuracy, resulting in essential improvements in computer technology such as improved fluctuation detection accuracy, increased early risk detection rate, and efficient data management. Furthermore, by linking multiple AI modules such as Transformers for difference extraction, fully connected networks for fluctuation classification, and softmax layers for probability estimation, the analysis unit achieves both detection accuracy for each fluctuation type and overall optimization. Application fields include elderly care facilities, home health management, rehabilitation support, and remote medical monitoring, and the system demonstrates significant technical effects especially in sites where labor shortages and limitations in observation accuracy are issues.

The evaluation unit can analyze fluctuations in movement and evaluate potential risks. For example, the evaluation unit analyzes fluctuations in movement and evaluates potential risks. For example, the evaluation unit can evaluate decreased walking speed as a risk. The evaluation unit can also evaluate reduced food intake as a risk. Thus, by evaluating potential risks, the evaluation unit can propose appropriate countermeasures. Some or all of the above-described processing in the evaluation unit may be performed using generative AI or may be performed without using generative AI. For example, the evaluation unit can input motion fluctuation data into a generative AI, and the generative AI can evaluate risks. Specifically, the evaluation unit inputs output values such as motion fluctuation score, fluctuation type label, and fluctuation probability distribution received from the analysis unit into a risk evaluation neural network (e.g., multilayer perceptron with threshold determination logic) to calculate risk labels (e.g., “decreased walking,” “reduced food intake”) and risk probabilities (e.g., risk of decreased walking 0.72, risk of reduced food intake 0.65). Examples of data input to the AI by the evaluation unit include (1) motion fluctuation score, (2) fluctuation type label, (3) fluctuation probability distribution, and (4) past risk evaluation history. Outputs received from the AI by the evaluation unit include (1) risk label, (2) risk probability, and (3) risk importance score (e.g., 0.9=very high). The evaluation unit inputs these output values into a threshold determination unit, generates a warning signal when the threshold is exceeded, and transmits the risk type, importance, and recommended countermeasure category to the proposal unit. These processes, unlike conventional subjective risk evaluation by humans, perform quantitative and objective risk estimation by AI at high speed and high accuracy, resulting in essential improvements in computer technology such as improved risk evaluation accuracy, increased early risk detection rate, and reduced user burden. Furthermore, by linking multiple AI modules such as multilayer perceptrons for risk evaluation, threshold determination logic, and memory networks for history reference, the evaluation unit achieves both evaluation accuracy for each risk type and overall optimization. Application fields include elderly care facilities, home health management, rehabilitation support, and remote medical monitoring, and the system demonstrates significant technical effects especially in sites where labor shortages and limitations in observation accuracy are issues.

The proposal unit can recommend preventive healthcare or supplements when a high risk is determined. For example, if a high risk is determined, the proposal unit recommends preventive healthcare or supplements. For example, the proposal unit can propose exercise therapy when walking speed decreases. The proposal unit can also propose nutritional supplements when food intake decreases. Thus, by proposing appropriate countermeasures when a high risk is determined, the proposal unit contributes to maintaining the health of elderly persons. Some or all of the above-described processing in the proposal unit may be performed using generative AI or may be performed without using generative AI. For example, the proposal unit can input risk evaluation data into a generative AI, and the generative AI can propose countermeasures. Specifically, the proposal unit inputs output values such as risk label, risk probability, and risk importance score received from the evaluation unit into a countermeasure generation large language model (e.g., LLM-based proposal generator) to generate personalized countermeasure plans (e.g., “recommend light walking three times a week,” “recommend intake of protein supplements”) considering the user's health status, past countermeasure history, and preference information. Examples of data input to the AI by the proposal unit include (1) risk label, (2) risk probability, (3) risk importance score, (4) user's health status, (5) past countermeasure history, and (6) preference information. Outputs received from the AI by the proposal unit include (1) recommended countermeasure text (e.g., “recommend light walking three times a week”), (2) recommended countermeasure category (e.g., “exercise therapy,” “nutritional supplement”), and (3) recommended countermeasure priority score (e.g., 0.95=highest priority). The proposal unit displays these output values on the user interface so that the user can easily implement the countermeasures. These processes, unlike conventional countermeasure proposals based on empirical rules or subjective judgment by humans, perform objective and personalized countermeasure generation by AI at high speed and high accuracy, resulting in essential improvements in computer technology such as improved countermeasure proposal accuracy, increased user satisfaction, and efficient health maintenance support. Furthermore, by linking multiple AI modules such as large language models for countermeasure generation, memory networks for history reference, and submodules for preference estimation, the proposal unit achieves both optimization for each user and overall optimization. Application fields include elderly care facilities, home health management, rehabilitation support, and remote medical monitoring, and the system demonstrates significant technical effects especially in sites where labor shortages and limitations in observation accuracy are issues.

The recording unit can estimate a user's emotion and adjust the level of detail of the data to be recorded based on the estimated emotion. For example, if the user is feeling stressed, the recording unit records detailed data to identify the cause of the stress. For example, the recording unit captures the user's facial expressions with a camera and estimates emotion using an emotion estimation algorithm. If the user is relaxed, the recording unit can record only basic data to reduce the burden. For example, the recording unit records the user's voice and estimates emotion using voice analysis technology. If the user is tired, the recording unit can reduce the recording frequency and record only the minimum necessary data. For example, the recording unit collects the user's biometric data (heart rate and skin conductance) with sensors and estimates emotion using an emotion estimation algorithm. Thus, by adjusting the level of detail of the data to be recorded according to the user's emotion, the recording unit can record more appropriate data. Emotion estimation is realized, for example, by using an emotion engine or generative AI with emotion estimation functions. Generative AI may be a text generation AI (e.g., LLM) or a multimodal generative AI, but is not limited to these examples. Some or all of the above-described processing in the recording unit may be performed using AI or may be performed without using AI. For example, the recording unit can input image data of the user captured by a camera into a generative AI, and the generative AI can estimate emotion. Specifically, the recording unit is equipped with multiple high-resolution cameras, a microphone array, and biometric sensors (heart rate, skin conductance, skin temperature, etc.), and simultaneously acquires facial images of the user (e.g., 30 frames×224×224×3), voice waveforms (e.g., 5 seconds of audio sampled at 16 kHz), and time-series biometric signals (e.g., 5 seconds of heart rate and skin conductance samples). The recording unit performs noise removal, normalization, and feature extraction (e.g., facial expression feature vectors, voice spectral features, heart rate variability indices) on these multimodal data in a preprocessing unit, and inputs the obtained feature vectors (e.g., facial expression 128 dimensions, voice 64 dimensions, biometric 32 dimensions) into a multimodal emotion estimation neural network (e.g., an architecture integrating CNN for images, RNN for voice, and MLP for biometrics). The emotion estimation neural network integrates features from each modality using a self-attention mechanism and outputs emotion state labels (e.g., “stress,” “relaxation,” “fatigue”) and emotion intensity scores (e.g., stress 0.82, relaxation 0.15). Examples of input to the AI include (1) facial image tensors (30×224×224×3), (2) voice waveforms (5 seconds of PCM data), and (3) time-series arrays of heart rate and skin conductance (5 seconds of sample values). Outputs from the AI include (1) emotion state label (e.g., “stress”), (2) emotion intensity score (e.g., stress 0.82), and (3) estimated basis features (e.g., contraction degree of depressor anguli oris muscle, voice pitch variation, heart rate variability index). Based on these output values, the recording unit applies a recording data detail control logic (e.g., if stress intensity is 0.7 or higher, record in detail every minute; if less than 0.3, record summary every 10 minutes) to automatically adjust recording frequency, recording items, and recording granularity. For example, if the stress state is high, posture during walking, eating motions, conversation content, and biometric signals are recorded frequently and in detail; if the relaxation state is present, only major health indicators are recorded at low frequency. These processes, unlike conventional subjective observation and uniform recording by humans, automate and optimize emotion estimation and recording control in high-dimensional feature spaces using multimodal AI, resulting in essential improvements in computer technology such as improved recording accuracy, reduced user burden, efficient data storage, and early identification of stress factors. Furthermore, by linking multiple AI modules such as multimodal neural networks for emotion estimation, detail control logic, and recording database management modules, the recording unit achieves flexible and highly accurate recording according to the user's state. Application fields include elderly care facilities, home health management, mental health monitoring, stress management support, and remote medical monitoring, and the system demonstrates significant technical effects especially in sites where emotional fluctuations are directly linked to health risks.

The recording unit can select an appropriate recording method by referring to the user's past health data at the time of recording. For example, the recording unit analyzes the user's past health data and sets the optimal recording frequency. For example, the recording unit refers to past diagnostic results and health checkup data and adjusts the recording frequency. The recording unit can also focus on recording specific movements based on the user's past health data. For example, the recording unit focuses on recording posture and speed during walking based on past health data. The recording unit can also select the type of data to be recorded based on the user's past health data. For example, the recording unit records the content and frequency of meals based on past health data. Thus, by referring to past health data, the recording unit can select the optimal recording method. Some or all of the above-described processing in the recording unit may be performed using AI or may be performed without using AI. For example, the recording unit can input past health data into a generative AI, and the generative AI can select the optimal recording method. Specifically, the recording unit is equipped with a database that manages time-series health checkup data accumulated for each user (e.g., structured database of blood pressure, heart rate, body weight, blood test values, walking speed, meal records), past medical diagnostic results (e.g., diagnostic labels, treatment history, medication history), and past recording frequency and item history. The recording unit performs normalization, missing value completion, and time-series feature extraction (e.g., moving average, trend score, anomaly detection) on these past data in a preprocessing unit, and inputs the obtained feature vectors (e.g., time-series array of health indicators for the past year, 365×10 dimensions) into a recording method optimization neural network (e.g., LSTM for time-series analysis+MLP for recording frequency control). The neural network learns patterns of past health status fluctuations, history of abnormal occurrences, and correlations between recording frequency and health event occurrence, and outputs the optimal recording frequency (e.g., once a day, every hour, only when events occur), recording items (e.g., walking posture, meal content, sleep, biometric signals), and recording granularity (e.g., detailed recording, summary recording). Examples of input to the AI include (1) time-series arrays of blood pressure, heart rate, and body weight for the past year (365×3 dimensions), (2) history of diagnostic labels (e.g., time-series of hypertension, diabetes), and (3) history of recording frequency and items (e.g., daily walking record, weekly meal record). Outputs from the AI include (1) recommended recording frequency (e.g., twice a day), (2) recommended recording item list (e.g., “walking posture,” “meal content”), and (3) recommended recording granularity (e.g., “detailed”). The recording unit inputs these output values into a recording control logic and automatically sets an optimized recording schedule, recording items, and recording granularity for each user. For example, for users with frequent decreases in walking speed in the past, walking posture and speed are recorded frequently and in detail, and for users with many abnormalities in meal content, meal recording is emphasized. These processes, unlike conventional uniform recording or recording settings based on human empirical rules, automate time-series health data analysis and recording method optimization by AI, resulting in essential improvements in computer technology such as improved recording accuracy, increased anomaly detection rate, efficient data storage, and reduced user burden. Furthermore, by linking multiple AI modules such as LSTM for time-series analysis, MLP for recording frequency control, and recording database management modules, the recording unit achieves flexible and highly accurate recording according to each user's health status and risk profile. Application fields include elderly care facilities, home health management, chronic disease monitoring, rehabilitation support, and remote medical monitoring, and the system demonstrates significant technical effects especially in sites where individual optimization is required.

The recording unit can adjust the recording frequency based on the user's living environment or activity level at the time of recording. For example, if the user's living environment changes, the recording unit adjusts the recording frequency. For example, the recording unit adjusts the recording frequency considering the housing environment, surrounding noise level, and climate conditions. If the user's activity level is high, the recording unit can increase the recording frequency. For example, the recording unit adjusts the recording frequency based on daily exercise amount, work content, and hobby activities. If the user's activity level is low, the recording unit can decrease the recording frequency. For example, if the user's activity amount is low, the recording unit records only the minimum necessary data. Thus, by adjusting the recording frequency according to the living environment and activity level, the recording unit can record appropriate data. Some or all of the above-described processing in the recording unit may be performed using AI or may be performed without using AI. For example, the recording unit can input data on the user's living environment and activity level into a generative AI, and the generative AI can adjust the recording frequency. Specifically, the recording unit is equipped with functions to acquire environmental sensor data (temperature, humidity, illuminance, noise level, etc.), accelerometer and gyroscope data (for activity measurement), and structured data on the user's living environment (housing type, number of cohabitants, surrounding environment, etc.). The recording unit performs normalization and feature extraction (e.g., hourly averages of temperature, humidity, noise level, daily steps, activity intensity score) on these environmental and activity data in a preprocessing unit, and inputs the obtained feature vectors (e.g., 24 hours×6 items of environmental and activity features) into a recording frequency optimization neural network (e.g., MLP for environmental features+LSTM for activity time series). The neural network learns correlations between changes in living environment, changes in activity level, and health event occurrence, and outputs the optimal recording frequency (e.g., hourly on high activity days, once a day on low activity days). Examples of input to the AI include (1) time-series arrays of temperature, humidity, illuminance, and noise level for one day (24×4 dimensions), (2) daily steps and activity intensity score (24×2 dimensions), and (3) housing environment information (e.g., detached house, apartment, number of cohabitants as categorical data). Outputs from the AI include (1) recommended recording frequency (e.g., hourly), and (2) recommended recording items (e.g., “activity amount,” “sleep,” “meals”). The recording unit inputs these output values into a recording control logic and automatically adjusts recording frequency and items according to the living environment and activity level. For example, on days with high noise levels or high activity, detailed recording is increased, and on days with quiet environments or low activity, recording frequency is decreased. These processes, unlike conventional uniform recording or subjective judgment by humans, automate analysis of environmental and activity data in high-dimensional feature spaces by AI and optimize recording frequency, resulting in essential improvements in computer technology such as improved recording accuracy, efficient data storage, reduced user burden, and improved responsiveness to environmental changes. Furthermore, by linking multiple AI modules such as MLP for environmental feature extraction, LSTM for activity analysis, and recording frequency control logic, the recording unit achieves flexible and highly accurate recording according to each user's living environment and activity level. Application fields include elderly care facilities, home health management, rehabilitation support, remote medical monitoring, and sites where changes in living environment affect health, and the system demonstrates significant technical effects especially in situations where environment-adaptive health monitoring is required.

The recording unit can estimate a user's emotion and determine the priority of data to be recorded based on the estimated emotion. For example, if the user is feeling stressed, the recording unit prioritizes recording data related to stress. For example, the recording unit captures the user's facial expressions with a camera and estimates emotion using an emotion estimation algorithm. If the user is relaxed, the recording unit can prioritize recording basic health data. For example, the recording unit records the user's voice and estimates emotion using voice analysis technology. If the user is tired, the recording unit can prioritize recording data related to fatigue. For example, the recording unit collects the user's biometric data (heart rate and skin conductance) with sensors and estimates emotion using an emotion estimation algorithm. Thus, by determining the priority of data to be recorded according to the user's emotion, the recording unit can prioritize recording important data. Emotion estimation is realized, for example, by using an emotion engine or generative AI with emotion estimation functions. Generative AI may be a text generation AI (e.g., LLM) or a multimodal generative AI, but is not limited to these examples. Some or all of the above-described processing in the recording unit may be performed using AI or may be performed without using AI. For example, the recording unit can input image data of the user captured by a camera into a generative AI, and the generative AI can estimate emotion. Specifically, the recording unit is equipped with multiple high-resolution cameras, a microphone array, and biometric sensors (heart rate, skin conductance, skin temperature, etc.), and simultaneously acquires facial images of the user (e.g., 30 frames×224×224×3), voice waveforms (e.g., 5 seconds of audio sampled at 16 kHz), and time-series biometric signals (e.g., 5 seconds of heart rate and skin conductance samples). The recording unit performs noise removal, normalization, and feature extraction (e.g., facial expression feature vectors, voice spectral features, heart rate variability indices) on these multimodal data in a preprocessing unit, and inputs the obtained feature vectors (e.g., facial expression 128 dimensions, voice 64 dimensions, biometric 32 dimensions) into a multimodal emotion estimation neural network (e.g., an architecture integrating CNN for images, RNN for voice, and MLP for biometrics). The emotion estimation neural network integrates features from each modality using a self-attention mechanism and outputs emotion state labels (e.g., “stress,” “relaxation,” “fatigue”) and emotion intensity scores (e.g., stress 0.82, relaxation 0.15). Examples of input to the AI include (1) facial image tensors (30×224×224×3), (2) voice waveforms (5 seconds of PCM data), and (3) time-series arrays of heart rate and skin conductance (5 seconds of sample values). Outputs from the AI include (1) emotion state label (e.g., “stress”), (2) emotion intensity score (e.g., stress 0.82), and (3) estimated basis features (e.g., contraction degree of depressor anguli oris muscle, voice pitch variation, heart rate variability index). Based on these output values, the recording unit applies a recording data priority determination logic (e.g., if stress intensity is 0.7 or higher, prioritize stress-related data; if relaxed, prioritize basic health indicators; if fatigued, prioritize biometric and rest-related data) to automatically adjust selection, order, and granularity of data to be recorded. For example, if the stress state is high, data directly related to stress factors such as heart rate variability, skin conductance, facial expression changes, and conversation content are prioritized for recording, and if relaxed, basic health indicators such as walking posture and meal content are prioritized. These processes, unlike conventional subjective observation and uniform recording by humans, automate and optimize emotion estimation and recording priority control in high-dimensional feature spaces using multimodal AI, resulting in essential improvements in computer technology such as improved recording accuracy, reduced user burden, efficient data storage, and early identification of stress factors. Furthermore, by linking multiple AI modules such as multimodal neural networks for emotion estimation, priority determination logic, and recording database management modules, the recording unit achieves flexible and highly accurate recording according to the user's state. Application fields include elderly care facilities, home health management, mental health monitoring, stress management support, and remote medical monitoring, and the system demonstrates significant technical effects especially in sites where emotional fluctuations are directly linked to health risks.

The recording unit can preferentially record highly relevant data based on the user's geographic location information at the time of recording. For example, if the user is in a specific location, the recording unit prioritizes recording data related to that location. For example, the recording unit acquires the user's geographic location information and records health information related to that location. If the user is traveling, the recording unit can prioritize recording travel-related data. For example, the recording unit acquires the user's travel destination geographic location information and records health information related to travel. If the user is at home, the recording unit can prioritize recording data related to daily life. For example, the recording unit acquires the user's home geographic location information and records health information related to daily life. Thus, by preferentially recording highly relevant data based on geographic location information, the recording unit can collect more useful data. Some or all of the above-described processing in the recording unit may be performed using AI or may be performed without using AI. For example, the recording unit can input the user's geographic location information into a generative AI, and the generative AI can preferentially record highly relevant data. Specifically, the recording unit acquires the user's geographic location information (e.g., latitude, longitude, altitude, indoor coordinates, location labels) in real time using GPS modules, Wi-Fi/Bluetooth beacons, indoor location estimation sensors, etc. The recording unit performs normalization, geographic clustering, and location labeling (e.g., home, workplace, hospital, travel destination) on these location data in a preprocessing unit, and inputs the obtained feature vectors (e.g., current location label+movement history vector for the past 24 hours) into a location-dependent recording optimization neural network (e.g., MLP for location features+LSTM for time-series movement history). The neural network learns correlations between health risks, behavior patterns, and recording priority items for each location, and outputs a recording priority list according to the current location (e.g., prioritize physical condition changes, meal content, and movement amount at travel destinations; prioritize daily life indicators at home). Examples of input to the AI include (1) latitude, longitude, and location label of the current location, (2) movement history vector for the past 24 hours (24×3 dimensions), and (3) history of health event occurrence for each location. Outputs from the AI include (1) recommended recording priority list (e.g., “travel destination: physical condition changes>meal content>movement amount,” “home: sleep>meal>exercise”), and (2) recommended recording items (e.g., “travel destination: blood pressure, body temperature, steps,” “home: sleep, meal”). The recording unit inputs these output values into a recording control logic and automatically adjusts selection, priority, and granularity of data to be recorded according to the current location and movement status. For example, during travel, physical condition changes and meal content are recorded frequently and in detail, and at home, daily life indicators are recorded regularly. These processes, unlike conventional uniform recording or subjective judgment by humans, automate analysis of geographic location information in high-dimensional feature spaces by AI and optimize recording priority, resulting in essential improvements in computer technology such as improved recording accuracy, increased anomaly detection rate, efficient data storage, and reduced user burden. Furthermore, by linking multiple AI modules such as MLP for location feature extraction, LSTM for movement history analysis, and recording priority control logic, the recording unit achieves flexible and highly accurate recording according to the user's geographic location and behavior patterns. Application fields include elderly care facilities, home health management, traveler health monitoring, remote medical monitoring, and sites where changes in living environment affect health, and the system demonstrates significant technical effects especially in situations where movement or environmental changes are directly linked to health risks.

The recording unit can analyze the user's social media activity and record relevant data at the time of recording. For example, if the user is highly active on social media, the recording unit records data related to that activity. For example, the recording unit analyzes the user's social media posts, number of likes, number of followers, etc., and records relevant data. If the user is less active on social media, the recording unit can prioritize recording other data. For example, if the user's social media activity is low, the recording unit prioritizes recording data related to daily life. The recording unit can also analyze the user's social media activity and record health-related data. For example, the recording unit analyzes health information and interests on the user's social media and records relevant data. Thus, by analyzing social media activity, the recording unit can record relevant data. Some or all of the above-described processing in the recording unit may be performed using AI or may be performed without using AI. For example, the recording unit can input the user's social media activity data into a generative AI, and the generative AI can record relevant data. Specifically, the recording unit collects structured and unstructured data from the user's social media accounts, such as post text, images, videos, number of likes, number of followers, comment history, frequency of health-related hashtags and keywords, etc. The recording unit performs text normalization, image feature extraction, and time-series aggregation (e.g., daily number of posts, number of reactions, proportion of health-related posts) on these data in a preprocessing unit, and inputs the obtained feature vectors (e.g., post text embedding 128 dimensions, image features 64 dimensions, activity score, health-related interest score) into a social media activity analysis neural network (e.g., an architecture integrating Transformer for text, CNN for images, and LSTM for time series). The neural network learns correlations between social activity patterns, health interest, activity fluctuations, and health event occurrence, and outputs a recording priority list (e.g., prioritize health indicators when health-related posts are frequent, prioritize daily life indicators when activity is low) and recommended recording items (e.g., record physical condition, meals, and exercise in detail when health-related posts are made). Examples of input to the AI include (1) embedding vectors of post text for the past week (7×128 dimensions), (2) image feature vectors (7×64 dimensions), and (3) daily number of posts, number of likes, proportion of health-related posts. Outputs from the AI include (1) recommended recording priority list (e.g., “health-related posts: physical condition >meals >exercise,” “activity decrease: sleep >daily life”), and (2) recommended recording items (e.g., “health-related posts: blood pressure, body temperature, meal content”). The recording unit inputs these output values into a recording control logic and automatically adjusts selection, priority, and granularity of data to be recorded according to social media activity status. For example, when health-related posts increase, physical condition, meals, and exercise are recorded frequently and in detail, and when activity decreases, sleep and daily life indicators are emphasized. These processes, unlike conventional uniform recording or subjective judgment by humans, automate analysis of social media activity in high-dimensional feature spaces by AI and optimize recording priority, resulting in essential improvements in computer technology such as improved recording accuracy, increased anomaly detection rate, efficient data storage, and reduced user burden. Furthermore, by linking multiple AI modules such as Transformer for text analysis, CNN for image feature extraction, LSTM for activity analysis, and recording priority control logic, the recording unit achieves flexible and highly accurate recording according to the user's social activity and health interest. Application fields include elderly care facilities, home health management, mental health monitoring, health behavior change support, and remote medical monitoring, and the system demonstrates significant technical effects especially in sites where social activity is directly linked to health risks or behavior change.

The comparison unit can estimate a user's emotion and adjust the comparison criteria based on the estimated emotion. For example, if the user is feeling stressed, the comparison unit focuses on comparing data related to stress. For example, the comparison unit captures the user's facial expressions with a camera and estimates emotion using an emotion estimation algorithm. If the user is relaxed, the comparison unit can compare basic health data. For example, the comparison unit records the user's voice and estimates emotion using voice analysis technology. If the user is tired, the comparison unit can focus on comparing data related to fatigue. For example, the comparison unit collects the user's biometric data (heart rate and skin conductance) with sensors and estimates emotion using an emotion estimation algorithm. Thus, by adjusting the comparison criteria according to the user's emotion, the comparison unit can perform more appropriate comparisons. Emotion estimation is realized, for example, by using an emotion engine or generative AI with emotion estimation functions. Generative AI may be a text generation AI (e.g., LLM) or a multimodal generative AI, but is not limited to these examples. Some or all of the above-described processing in the comparison unit may be performed using AI or may be performed without using AI. For example, the comparison unit can input image data of the user captured by a camera into a generative AI, and the generative AI can estimate emotion. Specifically, the comparison unit is equipped with multiple high-resolution cameras, a microphone array, and biometric sensors (heart rate, skin conductance, skin temperature, etc.), and simultaneously acquires facial images of the user (e.g., 30 frames×224×224×3), voice waveforms (5 seconds of audio sampled at 16 kHz), and time-series biometric signals (5 seconds of heart rate and skin conductance samples). The comparison unit performs noise removal, normalization, and feature extraction (e.g., facial expression feature vectors, voice spectral features, heart rate variability indices) on these multimodal data in a preprocessing unit, and inputs the obtained feature vectors (e.g., facial expression 128 dimensions, voice 64 dimensions, biometric 32 dimensions) into a multimodal emotion estimation neural network (e.g., an architecture integrating CNN for images, RNN for voice, and MLP for biometrics). The emotion estimation neural network integrates features from each modality using a self-attention mechanism and outputs emotion state labels (e.g., “stress,” “relaxation,” “fatigue”) and emotion intensity scores (e.g., stress 0.82, relaxation 0.15). Examples of input to the AI include (1) facial image tensors (30×224×224×3), (2) voice waveforms (5 seconds of PCM data), and (3) time-series arrays of heart rate and skin conductance (5 seconds of sample values). Outputs from the AI include (1) emotion state label (e.g., “stress”), (2) emotion intensity score (e.g., stress 0.82), and (3) estimated basis features (e.g., contraction degree of depressor anguli oris muscle, voice pitch variation, heart rate variability index). Based on these output values, the comparison unit applies a comparison criteria adjustment logic (e.g., if stress intensity is 0.7 or higher, focus on stress-related data; if relaxed, compare basic health indicators; if fatigued, focus on biometric and rest-related data) to automatically adjust selection, order, and granularity of data to be compared. For example, if the stress state is high, data directly related to stress factors such as heart rate variability, skin conductance, facial expression changes, and conversation content are focused on for comparison, and if relaxed, basic health indicators such as walking posture and meal content are compared. These processes, unlike conventional subjective observation and uniform comparison by humans, automate and optimize emotion estimation and comparison criteria control in high-dimensional feature spaces using multimodal AI, resulting in essential improvements in computer technology such as improved comparison accuracy, reduced user burden, efficient data storage, and early identification of stress factors. Furthermore, by linking multiple AI modules such as multimodal neural networks for emotion estimation, comparison criteria adjustment logic, and comparison database management modules, the comparison unit achieves flexible and highly accurate comparison according to the user's state. Application fields include elderly care facilities, home health management, mental health monitoring, stress management support, and remote medical monitoring, and the system demonstrates significant technical effects especially in sites where emotional fluctuations are directly linked to health risks.

The comparison unit can appropriately select a comparison algorithm by referring to past comparison data at the time of comparison. For example, the comparison unit analyzes past comparison data and selects the optimal comparison algorithm. For example, the comparison unit adjusts the data comparison method or algorithm parameters based on past comparison data. The comparison unit can also adjust the comparison algorithm based on past comparison data. For example, the comparison unit refers to past comparison data and optimizes the algorithm to improve comparison accuracy. Thus, by referring to past comparison data, the comparison unit can optimize the comparison algorithm. Some or all of the above-described processing in the comparison unit may be performed using AI or may be performed without using AI. For example, the comparison unit can input past comparison data into a generative AI, and the generative AI can optimize the comparison algorithm. Specifically, the comparison unit is equipped with a database that manages time-series comparison history data accumulated for each user (e.g., past one year of comparison result scores, feature vectors of comparison target data, algorithm parameter history). The comparison unit performs normalization, missing value completion, and time-series feature extraction (e.g., moving average, trend score, anomaly detection) on these past comparison data in a preprocessing unit, and inputs the obtained feature vectors (e.g., comparison history array of 365×10 dimensions) into a comparison algorithm optimization neural network (e.g., LSTM for time-series analysis+MLP for algorithm parameter control). The neural network learns patterns of past comparison accuracy fluctuations, history of anomaly occurrences, and correlations between algorithm parameters and comparison accuracy, and outputs the optimal comparison algorithm (e.g., tensor similarity calculation, dynamic time warping, feature space clustering), parameter set (e.g., threshold, weighting coefficient, comparison target selection rule). Examples of input to the AI include (1) time-series array of comparison scores for the past year (365×3dimensions), (2) history of algorithm parameters (e.g., time-series of threshold 0.7, weight 0.5), and (3) anomaly detection history (e.g., list of misjudgment occurrence dates). Outputs from the AI include (1) recommended comparison algorithm (e.g., “dynamic time warping”), (2) recommended parameter set (e.g., threshold 0.75, weight 0.6), and (3) comparison accuracy prediction score (e.g., 0.92). The comparison unit inputs these output values into a comparison control logic and automatically sets optimized comparison algorithms and parameters for each user and data type. For example, if there were many misjudgments in walking comparison in the past, the algorithm is changed, and if accuracy was high in meal comparison, the current parameters are maintained. These processes, unlike conventional uniform algorithms or comparison settings based on human empirical rules, automate time-series comparison data analysis and algorithm optimization by AI, resulting in essential improvements in computer technology such as improved comparison accuracy, increased anomaly detection rate, efficient data storage, and reduced user burden. Furthermore, by linking multiple AI modules such as LSTM for time-series analysis, MLP for algorithm parameter control, and comparison database management modules, the comparison unit achieves flexible and highly accurate comparison according to each user's comparison history and risk profile. Application fields include elderly care facilities, home health management, chronic disease monitoring, rehabilitation support, and remote medical monitoring, and the system demonstrates significant technical effects especially in sites where individual optimization is required.

The comparison unit can adjust the comparison frequency based on the user's lifestyle or activity pattern at the time of comparison. For example, if the user's lifestyle changes, the comparison unit adjusts the comparison frequency. For example, the comparison unit adjusts the comparison frequency based on meal patterns, exercise habits, and sleep habits. If the user's activity pattern changes, the comparison unit can also adjust the comparison frequency. For example, the comparison unit adjusts the comparison frequency based on daily exercise amount, work content, and hobby activities. The comparison unit can also optimize the comparison frequency based on the user's lifestyle and activity pattern. For example, the comparison unit adjusts the comparison frequency based on the user's lifestyle and activity pattern. Thus, by adjusting the comparison frequency according to lifestyle and activity pattern, the comparison unit can perform appropriate comparisons. Some or all of the above-described processing in the comparison unit may be performed using AI or may be performed without using AI. For example, the comparison unit can input data on the user's lifestyle and activity pattern into a generative AI, and the generative AI can adjust the comparison frequency. Specifically, the comparison unit is equipped with a database that manages lifestyle data (e.g., time-series data of meal records, exercise records, sleep records), activity pattern data (e.g., daily steps, activity intensity, hobby activity logs), and past comparison frequency and result history. The comparison unit performs normalization and feature extraction (e.g., weekly average activity amount, meal pattern, sleep rhythm) on these data in a preprocessing unit, and inputs the obtained feature vectors (e.g., 7 days×10 items of lifestyle and activity features) into a comparison frequency optimization neural network (e.g., MLP for activity features+LSTM for time series). The neural network learns correlations between lifestyle and activity pattern fluctuations, comparison accuracy, and health event occurrence, and outputs the optimal comparison frequency (e.g., daily on high activity weeks, weekly on low activity weeks). Examples of input to the AI include (1) time-series array of meal, exercise, and sleep records for one week (7×3 dimensions), (2) weekly activity intensity score (7×1 dimension), and (3) history of comparison frequency and results (e.g., daily comparison, weekly comparison). Outputs from the AI include (1) recommended comparison frequency (e.g., daily), and (2) recommended comparison items (e.g., “exercise,” “sleep”). The comparison unit inputs these output values into a comparison control logic and automatically adjusts comparison frequency and items according to lifestyle and activity pattern. For example, if activity increases in a week, comparison frequency is increased, and if activity decreases, comparison frequency is decreased. These processes, unlike conventional uniform comparison or subjective judgment by humans, automate analysis of lifestyle and activity data in high-dimensional feature spaces by AI and optimize comparison frequency, resulting in essential improvements in computer technology such as improved comparison accuracy, efficient data storage, reduced user burden, and improved responsiveness to lifestyle changes. Furthermore, by linking multiple AI modules such as MLP for activity feature extraction, LSTM for time-series analysis, and comparison frequency control logic, the comparison unit achieves flexible and highly accurate comparison according to each user's lifestyle and activity pattern. Application fields include elderly care facilities, home health management, rehabilitation support, remote medical monitoring, and sites where changes in lifestyle affect health, and the system demonstrates significant technical effects especially in situations where lifestyle-adaptive health monitoring is required.

The comparison unit can estimate a user's emotion and adjust the display method of comparison results based on the estimated emotion. For example, if the user is feeling stressed, the comparison unit provides a simple and highly visible display method. For example, the comparison unit captures the user's facial expressions with a camera and estimates emotion using an emotion estimation algorithm. If the user is relaxed, the comparison unit can provide a display method that includes detailed information. For example, the comparison unit records the user's voice and estimates emotion using voice analysis technology. If the user is tired, the comparison unit can provide a display method that highlights key points. For example, the comparison unit collects the user's biometric data (heart rate and skin conductance) with sensors and estimates emotion using an emotion estimation algorithm. Thus, by adjusting the display method of comparison results according to the user's emotion, the comparison unit can provide more appropriate displays. Emotion estimation is realized, for example, by using an emotion engine or generative AI with emotion estimation functions. Generative AI may be a text generation AI (e.g., LLM) or a multimodal generative AI, but is not limited to these examples. Some or all of the above-described processing in the comparison unit may be performed using AI or may be performed without using AI. For example, the comparison unit can input image data of the user captured by a camera into a generative AI, and the generative AI can estimate emotion. Specifically, the comparison unit is equipped with multiple high-resolution cameras, a microphone array, and biometric sensors (heart rate, skin conductance, skin temperature, etc.), and simultaneously acquires facial images of the user (e.g., 30 frames×224×224×3), voice waveforms (5 seconds of audio sampled at 16 kHz), and time-series biometric signals (5 seconds of heart rate and skin conductance samples). The comparison unit performs noise removal, normalization, and feature extraction (e.g., facial expression feature vectors, voice spectral features, heart rate variability indices) on these multimodal data in a preprocessing unit, and inputs the obtained feature vectors (e.g., facial expression 128dimensions, voice 64 dimensions, biometric 32 dimensions) into a multimodal emotion estimation neural network (e.g., an architecture integrating CNN for images, RNN for voice, and MLP for biometrics). The emotion estimation neural network integrates features from each modality using a self-attention mechanism and outputs emotion state labels (e.g., “stress,” “relaxation,” “fatigue”) and emotion intensity scores (e.g., stress 0.82, relaxation 0.15). Examples of input to the AI include (1) facial image tensors (30×224×224×3), (2) voice waveforms (5 seconds of PCM data), and (3) time-series arrays of heart rate and skin conductance (5 seconds of sample values). Outputs from the AI include (1) emotion state label (e.g., “stress”), (2) emotion intensity score (e.g., stress 0.82), and (3) estimated basis features (e.g., contraction degree of depressor anguli oris muscle, voice pitch variation, heart rate variability index). Based on these output values, the comparison unit applies a display method control logic (e.g., if stress intensity is 0.7 or higher, use simple display; if relaxed, use detailed display; if fatigued, use key point display) to automatically adjust display content, display granularity, and display order of comparison results. For example, if the stress state is high, only major health indicators are displayed in large font and color coding, and if relaxed, detailed graphs, time-series fluctuations, and basis data are displayed. These processes, unlike conventional uniform display or subjective judgment by humans, automate and optimize emotion estimation and display control in high-dimensional feature spaces using multimodal AI, resulting in essential improvements in computer technology such as reduced cognitive burden on users, improved information transmission efficiency, and early identification of stress factors. Furthermore, by linking multiple AI modules such as multimodal neural networks for emotion estimation, display method control logic, and user interface management modules, the comparison unit achieves flexible and highly accurate display according to the user's state. Application fields include elderly care facilities, home health management, mental health monitoring, stress management support, and remote medical monitoring, and the system demonstrates significant technical effects especially in sites where emotional fluctuations are directly linked to health risks or information receptivity.

The comparison unit can perform comparison based on the user's geographic distribution at the time of comparison. For example, if the user is in a specific region, the comparison unit compares data related to that region. For example, the comparison unit considers the user's geographic distribution and compares health information related to that region. If the user is traveling, the comparison unit can compare travel destination data. For example, the comparison unit considers the user's geographic distribution at the travel destination and compares health information related to travel. The comparison unit can also select the optimal comparison method based on the user's geographic distribution. For example, the comparison unit optimizes the algorithm to improve comparison accuracy based on the user's geographic distribution. Thus, by performing comparison based on geographic distribution, the comparison unit can perform more appropriate comparisons. Some or all of the above-described processing in the comparison unit may be performed using AI or may be performed without using AI. For example, the comparison unit can input the user's geographic distribution data into a generative AI, and the generative AI can perform comparison. Specifically, the comparison unit acquires the user's geographic location information (e.g., latitude, longitude, altitude, indoor coordinates, location labels) in real time using GPS modules, Wi-Fi/Bluetooth beacons, indoor location estimation sensors, etc. The comparison unit performs normalization, geographic clustering, and location labeling (e.g., home, workplace, hospital, travel destination) on these location data in a preprocessing unit, and inputs the obtained feature vectors (e.g., current location label+movement history vector for the past 24 hours) into a location-dependent comparison optimization neural network (e.g., MLP for location features+LSTM for time-series movement history). The neural network learns correlations between health risks, behavior patterns, and comparison priority items for each location, and outputs a comparison priority list according to the current location (e.g., prioritize physical condition changes, meal content, and movement amount at travel destinations; prioritize daily life indicators at home) and the optimal comparison algorithm (e.g., automatically adjust threshold and weighting for each location). Examples of input to the AI include (1) latitude, longitude, and location label of the current location, (2) movement history vector for the past 24 hours (24×3 dimensions), and (3) history of health event occurrence for each location. Outputs from the AI include (1) recommended comparison priority list (e.g., “travel destination: physical condition changes >meal content >movement amount,” “home: sleep >meal >exercise”), (2) recommended comparison algorithm (e.g., “dynamic time warping”), and (3) recommended parameter set (e.g., threshold 0.75, weight 0.6). The comparison unit inputs these output values into a comparison control logic and automatically adjusts selection, order, comparison algorithm, and parameters of data to be compared according to the current location and movement status. For example, during travel, physical condition changes and meal content are focused on for comparison, and at home, daily life indicators are compared regularly. These processes, unlike conventional uniform comparison or subjective judgment by humans, automate analysis of geographic location information in high-dimensional feature spaces by AI and optimize comparison, resulting in essential improvements in computer technology such as improved comparison accuracy, increased anomaly detection rate, efficient data storage, and reduced user burden. Furthermore, by linking multiple AI modules such as MLP for location feature extraction, LSTM for movement history analysis, and comparison priority control logic, the comparison unit achieves flexible and highly accurate comparison according to the user's geographic location and behavior patterns. Application fields include elderly care facilities, home health management, traveler health monitoring, remote medical monitoring, and sites where changes in living environment affect health, and the system demonstrates significant technical effects especially in situations where movement or environmental changes are directly linked to health risks or comparison accuracy.

The comparison unit can refer to the user's related literature at the time of comparison to improve the accuracy of comparison. For example, the comparison unit refers to literature related to the user's health to improve comparison accuracy. For example, the comparison unit refers to academic papers, medical guidelines, and specialized books to improve comparison accuracy. The comparison unit can also refer to literature related to the user's lifestyle to improve comparison accuracy. For example, the comparison unit improves comparison accuracy based on literature related to the user's lifestyle. The comparison unit can also refer to literature related to the user's activity pattern to improve comparison accuracy. For example, the comparison unit improves comparison accuracy based on literature related to the user's activity pattern. Thus, by referring to related literature, the comparison unit can improve comparison accuracy. Some or all of the above-described processing in the comparison unit may be performed using AI or may be performed without using AI. For example, the comparison unit can input the user's related literature data into a generative AI, and the generative AI can improve comparison accuracy. Specifically, the comparison unit is configured to refer to a literature database containing academic papers, medical guidelines, specialized books, and the latest research results related to the user's health status, lifestyle, and activity patterns. The comparison unit performs text normalization, summarization, keyword extraction, and knowledge graph construction (e.g., extraction of relationships among disease names, symptoms, recommended countermeasures, risk factors) on these literature data in a preprocessing unit, and inputs the obtained knowledge vectors (e.g., literature summary embedding 256 dimensions, risk factor relevance score) into a literature-referenced comparison optimization neural network (e.g., Transformer for text+integrated layer for knowledge graph embedding). The neural network learns correlations between the user's health data, lifestyle, activity patterns, and literature knowledge, and outputs selection of comparison algorithms, parameter adjustment, and generation of explanation for comparison basis (e.g., “this comparison is based on XX guideline”). Examples of input to the AI include (1) literature summary vectors related to the user's health indicators, (2) knowledge graphs related to lifestyle and activity patterns, and (3) relevance scores between comparison target data and literature risk factors. Outputs from the AI include (1) recommended comparison algorithm (e.g., “guideline-compliant type”), (2) recommended parameter set (e.g., threshold 0.8, weight 0.7), and (3) explanation text for comparison basis (e.g., “emphasizing decreased walking speed based on XX paper”). The comparison unit inputs these output values into a comparison control logic and automatically sets comparison algorithms, parameters, and basis explanations based on literature knowledge. For example, indicators recommended in the latest medical guidelines are prioritized for comparison, and if the risk of lifestyle-related diseases is high, thresholds from related literature are applied. These processes, unlike conventional empirical rules or subjective judgment by humans, automate analysis of literature knowledge in high-dimensional feature spaces by AI and optimize comparison, resulting in essential improvements in computer technology such as improved comparison accuracy, improved explanation of basis, increased user reliability, and efficient data management. Furthermore, by linking multiple AI modules such as Transformer for text analysis, integrated layer for knowledge graph, and comparison basis explanation generation module, the comparison unit achieves flexible and highly accurate comparison according to each user's health status, lifestyle, and activity pattern. Application fields include elderly care facilities, home health management, chronic disease monitoring, rehabilitation support, remote medical monitoring, and evidence-based medical support, and the system demonstrates significant technical effects especially in sites where explanation of basis and accuracy are important.

The analysis unit can estimate a user's emotion and adjust the level of detail of analysis based on the estimated emotion. For example, when the user is feeling stressed, the analysis unit performs a detailed analysis to identify the cause of the stress. For instance, the analysis unit captures the user's facial expressions with a camera and estimates the emotion using an emotion estimation algorithm. Additionally, when the user is relaxed, the analysis unit can perform basic analysis to reduce the burden. For example, the analysis unit records the user's voice and estimates the emotion using voice analysis technology. Furthermore, when the user is fatigued, the analysis unit can reduce the frequency of analysis and perform only the minimum necessary analysis. For example, the analysis unit collects the user's biometric data (such as heart rate and skin conductance) with sensors and estimates the emotion using an emotion estimation algorithm. In this way, by adjusting the level of detail of analysis according to the user's emotion, the analysis unit enables more appropriate analysis. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the analysis unit may be performed using AI or without using AI. For example, the analysis unit may input user image data captured by a camera into generative AI, which then estimates the emotion. Specifically, the analysis unit is equipped with multiple high-resolution cameras, a microphone array, and biometric sensors (such as heart rate, skin conductance, skin temperature, etc.), and simultaneously acquires facial expression images (e.g., 30 frames×224×224×3), audio waveforms (5 seconds of audio sampled at 16 kHz), and biometric signal time series (5 seconds of heart rate and skin conductance samples). The analysis unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82, relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the analysis unit applies analysis detail control logic (e.g., detailed analysis if stress intensity is 0.7 or higher, summary analysis if below 0.3, reduced analysis frequency if in a fatigue state) to automatically adjust analysis frequency, analysis items, and analysis granularity. For example, when stress is high, posture during walking, eating behavior, conversation content, and biometric signals are analyzed in detail and at high frequency; when relaxed, only major health indicators are analyzed at low frequency. These processes, unlike conventional subjective observation or uniform analysis by humans, automate and optimize emotion estimation and analysis control in a high-dimensional feature space using multimodal AI, thereby fundamentally improving computer technology by enhancing analysis accuracy, reducing user burden, improving data storage efficiency, and enabling early identification of stress factors. Furthermore, the analysis unit achieves flexible and highly accurate analysis according to user state by linking multiple AI modules such as a multimodal neural network for emotion estimation, detail control logic, and analysis database management modules. Application fields include elderly care facilities, home health management, mental health monitoring, stress management support, and remote medical monitoring, and the technology is particularly effective in environments where emotional fluctuations are directly linked to health risks.

The analysis unit can refer to past analysis data during analysis to appropriately select the analysis algorithm. For example, the analysis unit analyzes past analysis data and selects the optimal analysis algorithm. The analysis unit may also adjust the analysis method or algorithm parameters based on past analysis data. Furthermore, the analysis unit can optimize the algorithm by referring to past analysis data to improve analysis accuracy. Thus, by referring to past analysis data, the analysis unit can optimize the analysis algorithm. Some or all of the above-described processing in the analysis unit may be performed using generative AI or without using generative AI. For example, the analysis unit may input past analysis data into generative AI, which then optimizes the analysis algorithm. Specifically, the analysis unit is equipped with a database that manages time-series accumulated analysis history data for each user (e.g., analysis result scores for the past year, feature vectors of analyzed data, algorithm parameter history, etc.). The analysis unit preprocesses these past analysis data in a preprocessing unit for normalization, missing value completion, and time-series feature extraction (e.g., moving average, trend score, anomaly detection), and inputs the obtained feature vectors (e.g., 365×10-dimensional analysis history array) into an analysis algorithm optimization neural network (e.g., LSTM for time-series analysis and MLP for algorithm parameter control). This neural network learns patterns of past analysis accuracy fluctuations, anomaly occurrence history, and correlations between algorithm parameters and analysis accuracy, and outputs the optimal analysis algorithm (e.g., tensor similarity calculation, dynamic time warping, feature space clustering), parameter sets (e.g., thresholds, weighting coefficients, analysis target selection rules), and analysis accuracy prediction scores. Examples of AI input include (1) time-series arrays of analysis scores for the past year (365×3 dimensions), (2) algorithm parameter history (e.g., time-series of threshold 0.7, weight 0.5, etc.), and (3) anomaly detection history (e.g., list of false positive dates). AI output includes (1) recommended analysis algorithm (e.g., “dynamic time warping”), (2) recommended parameter set (e.g., threshold 0.75, weight 0.6), and (3) analysis accuracy prediction score (e.g., 0.92). The analysis unit inputs these output values into analysis control logic to automatically set optimized analysis algorithms and parameters for each user and data type. For example, if there were many false positives in gait analysis in the past, the algorithm is changed; if accuracy was high in meal analysis, the current parameters are maintained. These processes, unlike conventional uniform algorithms or human experience-based analysis settings, automate time-series analysis data analysis and algorithm optimization using AI, thereby fundamentally improving computer technology by enhancing analysis accuracy, anomaly detection rate, data storage efficiency, and reducing user burden. Furthermore, the analysis unit achieves flexible and highly accurate analysis according to each user's analysis history and risk profile by linking multiple AI modules such as LSTM for time-series analysis, MLP for algorithm parameter control, and analysis database management modules. Application fields include elderly care facilities, home health management, chronic disease monitoring, rehabilitation support, and remote medical monitoring, and the technology is particularly effective in environments where individual optimization is required.

The analysis unit can adjust the frequency of analysis during analysis based on the user's health condition and lifestyle habits. For example, when the user's health condition changes, the analysis unit adjusts the frequency of analysis. The analysis unit may adjust the frequency of analysis based on health conditions such as blood pressure, heart rate, and body weight. Furthermore, when the user's lifestyle habits change, the analysis unit can also adjust the frequency of analysis. For example, the analysis unit adjusts the frequency of analysis based on meal patterns, exercise habits, and sleep habits. The analysis unit can also optimize the frequency of analysis based on the user's health condition and lifestyle habits. Thus, by adjusting the frequency of analysis according to health condition and lifestyle habits, the analysis unit enables appropriate analysis. Some or all of the above-described processing in the analysis unit may be performed using generative AI or without using generative AI. For example, the analysis unit may input data on the user's health condition and lifestyle habits into generative AI, which then adjusts the frequency of analysis. Specifically, the analysis unit is equipped with a database that manages the user's health condition data (e.g., time-series data of blood pressure, heart rate, body weight, blood test values, etc.), lifestyle habit data (e.g., meal records, exercise records, sleep records, etc.), and past analysis frequency and analysis result history. The analysis unit preprocesses these data in a preprocessing unit for normalization and feature extraction (e.g., weekly average health indicators, meal patterns, sleep rhythms, etc.), and inputs the obtained feature vectors (e.g., 7 days×10 health and lifestyle habit features) into an analysis frequency optimization neural network (e.g., MLP for health features and LSTM for time-series). This neural network learns the correlation between health condition/lifestyle habit fluctuations and analysis accuracy/health event occurrence, and outputs the optimal analysis frequency (e.g., once per day in weeks with unstable health, once per week in stable weeks). Examples of AI input include (1) time-series arrays of blood pressure, heart rate, and body weight for one week (7×3 dimensions), (2) one week of meal, exercise, and sleep records (7×3 dimensions), and (3) past analysis frequency and analysis result history (e.g., daily analysis, weekly analysis, etc.). AI output includes (1) recommended analysis frequency (e.g., once per day), and (2) recommended analysis items (e.g., “exercise”, “sleep”). The analysis unit inputs these output values into analysis control logic to automatically adjust analysis frequency and analysis items according to health condition and lifestyle habits. For example, when health condition worsens, analysis frequency is increased; when stable, analysis frequency is decreased. These processes, unlike conventional uniform analysis or subjective human judgment, automate analysis and analysis frequency optimization in a high-dimensional feature space of health and lifestyle habit data using AI, thereby fundamentally improving computer technology by enhancing analysis accuracy, data storage efficiency, reducing user burden, and improving responsiveness to health fluctuations. Furthermore, the analysis unit achieves flexible and highly accurate analysis according to each user's health condition and lifestyle habits by linking multiple AI modules such as MLP for health feature extraction, LSTM for time-series analysis, and analysis frequency control logic. Application fields include elderly care facilities, home health management, rehabilitation support, remote medical monitoring, and environments where health condition fluctuations affect health, and the technology is particularly effective in situations where health-adaptive monitoring is required.

The analysis unit can estimate a user's emotion and adjust the display method of analysis results based on the estimated emotion. For example, when the user is feeling stressed, the analysis unit provides a simple and highly visible display method. For instance, the analysis unit captures the user's facial expressions with a camera and estimates the emotion using an emotion estimation algorithm. Additionally, when the user is relaxed, the analysis unit can provide a display method that includes detailed information. For example, the analysis unit records the user's voice and estimates the emotion using voice analysis technology. Furthermore, when the user is fatigued, the analysis unit can provide a display method that highlights key points. For example, the analysis unit collects the user's biometric data (such as heart rate and skin conductance) with sensors and estimates the emotion using an emotion estimation algorithm. In this way, by adjusting the display method of analysis results according to the user's emotion, the analysis unit enables more appropriate display. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the analysis unit may be performed using AI or without using AI. For example, the analysis unit may input user image data captured by a camera into generative AI, which then estimates the emotion. Specifically, the analysis unit is equipped with multiple high-resolution cameras, a microphone array, and biometric sensors (such as heart rate, skin conductance, skin temperature, etc.), and simultaneously acquires facial expression images (e.g., 30 frames×224×224×3), audio waveforms (5 seconds of audio sampled at 16 kHz), and biometric signal time series (5 seconds of heart rate and skin conductance samples). The analysis unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82,relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the analysis unit applies display method control logic (e.g., simple display if stress intensity is 0.7 or higher, detailed display if relaxed, key point display if fatigued) to automatically adjust the display content, display granularity, and display order of analysis results. For example, when stress is high, only major health indicators are displayed in large font and color-coded; when relaxed, detailed graphs, time-series variations, and basis data are displayed. These processes, unlike conventional uniform display or subjective human judgment, automate and optimize emotion estimation and display control in a high-dimensional feature space using multimodal AI, thereby fundamentally improving computer technology by reducing user cognitive burden, improving information transmission efficiency, and enabling early identification of stress factors. Furthermore, the analysis unit achieves flexible and highly accurate display according to user state by linking multiple AI modules such as a multimodal neural network for emotion estimation, display method control logic, and user interface management modules. Application fields include elderly care facilities, home health management, mental health monitoring, stress management support, and remote medical monitoring, and the technology is particularly effective in environments where emotional fluctuations are directly linked to health risks and information receptivity.

The analysis unit can perform analysis during analysis based on the user's geographic distribution. For example, when the user is in a specific region, the analysis unit analyzes data related to that region. For instance, the analysis unit considers the user's geographic distribution and analyzes health information related to that region. Additionally, when the user is traveling, the analysis unit can analyze data related to the travel destination. For example, the analysis unit considers the user's geographic distribution at the travel destination and analyzes health information related to travel. Furthermore, the analysis unit can select the optimal analysis method based on the user's geographic distribution. For example, the analysis unit optimizes the algorithm based on the user's geographic distribution to improve analysis accuracy. Thus, by performing analysis based on geographic distribution, the analysis unit enables more appropriate analysis. Some or all of the above-described processing in the analysis unit may be performed using generative AI or without using generative AI. For example, the analysis unit may input the user's geographic distribution data into generative AI, which then performs the analysis. Specifically, the analysis unit acquires the user's geographic location information (e.g., latitude, longitude, altitude, indoor coordinates, location labels, etc.) in real time using GPS modules, Wi-Fi/Bluetooth beacons, indoor positioning sensors, etc. The analysis unit preprocesses these location data in a preprocessing unit for normalization, geographic clustering, and location labeling (e.g., home, workplace, hospital, travel destination, etc.), and inputs the obtained feature vectors (e.g., current location label plus past 24-hour movement history vector) into a location-dependent analysis optimization neural network (e.g., MLP for location features and LSTM for time-series movement history). This neural network learns the correlation between health risks, behavior patterns, and analysis priority items for each location, and outputs a location-based analysis priority list (e.g., prioritize physical condition changes, meal content, and movement amount at travel destinations; prioritize daily life indicators at home) and optimal analysis algorithms (e.g., automatically adjust thresholds and weights for each location). Examples of AI input include (1) current location latitude, longitude, and location label, (2) past 24-hour movement history vector (24×3 dimensions), and (3) health event occurrence history for each location. AI output includes (1) recommended analysis priority list (e.g., “Travel destination: physical condition change>meal content>movement amount”, “Home: sleep>meal>exercise”), (2) recommended analysis algorithm (e.g., “dynamic time warping”), and (3) recommended parameter set (e.g., threshold 0.75, weight 0.6). The analysis unit inputs these output values into analysis control logic to automatically adjust selection of analysis target data, analysis order, analysis algorithm, and parameters according to current location and movement status. For example, during travel, physical condition changes and meal content are analyzed with emphasis, while at home, daily life indicators are analyzed regularly. These processes, unlike conventional uniform analysis or subjective human judgment, automate and optimize analysis and analysis optimization in a high-dimensional feature space of geographic location information using AI, thereby fundamentally improving computer technology by enhancing analysis accuracy, anomaly detection rate, data storage efficiency, and reducing user burden. Furthermore, the analysis unit achieves flexible and highly accurate analysis according to the user's geographic location and behavior patterns by linking multiple AI modules such as MLP for location feature extraction, LSTM for movement history analysis, and analysis priority control logic. Application fields include elderly care facilities, home health management, traveler health monitoring, remote medical monitoring, and environments where changes in living environment affect health, and the technology is particularly effective in situations where movement or environmental changes are directly linked to health risks and analysis accuracy.

The analysis unit can refer to related literature during analysis to improve analysis accuracy. For example, the analysis unit refers to literature related to the user's health to improve analysis accuracy. The analysis unit may also refer to academic papers, medical guidelines, and specialized books to improve analysis accuracy. Furthermore, the analysis unit can refer to literature related to the user's lifestyle habits to improve analysis accuracy. For example, the analysis unit improves analysis accuracy based on literature related to the user's lifestyle habits. Additionally, the analysis unit can refer to literature related to the user's activity patterns to improve analysis accuracy. For example, the analysis unit improves analysis accuracy based on literature related to the user's activity patterns. Thus, by referring to related literature, the analysis unit can improve analysis accuracy. Some or all of the above-described processing in the analysis unit may be performed using generative AI or without using generative AI. For example, the analysis unit may input the user's related literature data into generative AI, which then improves analysis accuracy. Specifically, the analysis unit is configured to refer to a literature database containing academic papers, medical guidelines, specialized books, and the latest research results related to the user's health condition, lifestyle habits, and activity patterns. The analysis unit preprocesses these literature data in a preprocessing unit for text normalization, summarization, keyword extraction, and knowledge graph construction (e.g., extraction of relationships among disease names, symptoms, recommended countermeasures, and risk factors), and inputs the obtained knowledge vectors (e.g., 256-dimensional literature summary embeddings, risk factor relevance scores) into a literature-referenced analysis optimization neural network (e.g., Transformer for text and an integrated layer for knowledge graph embeddings). This neural network learns the correlation between the user's health data, lifestyle habits, activity patterns, and literature knowledge, and outputs selection of analysis algorithms, parameter adjustment, and generation of explanation texts for analysis rationale (e.g., “This analysis is based on XX guideline”). Examples of AI input include (1) literature summary vectors related to the user's health indicators, (2) knowledge graphs related to lifestyle habits and activity patterns, and (3) relevance scores between analysis target data and literature risk factors. AI output includes (1) recommended analysis algorithm (e.g., “guideline-compliant type”), (2) recommended parameter set (e.g., threshold 0.8, weight 0.7), and (3) analysis rationale explanation text (e.g., “Emphasis on decreased walking speed based on XX paper”). The analysis unit inputs these output values into analysis control logic to automatically set analysis algorithms, parameters, and rationale explanations based on literature knowledge. For example, indicators recommended in the latest medical guidelines are prioritized for analysis, and when the risk of lifestyle-related diseases is high, thresholds from related literature are applied. These processes, unlike conventional experience-based or subjective human judgment, automate and optimize analysis and analysis optimization in a high-dimensional feature space of literature knowledge using AI, thereby fundamentally improving computer technology by enhancing analysis accuracy, rationale explainability, user reliability, and data management efficiency. Furthermore, the analysis unit achieves flexible and highly accurate analysis according to each user's health condition, lifestyle habits, and activity patterns by linking multiple AI modules such as Transformer for text analysis, knowledge graph integration layers, and analysis rationale explanation generation modules. Application fields include elderly care facilities, home health management, chronic disease monitoring, rehabilitation support, remote medical monitoring, and evidence-based medical support, and the technology is particularly effective in environments where rationale explainability and accuracy are emphasized.

The evaluation unit can estimate a user's emotion and adjust the criteria for risk evaluation based on the estimated emotion. For example, when the user is feeling stressed, the evaluation unit prioritizes evaluation of risks related to stress. For instance, the evaluation unit captures the user's facial expressions with a camera and estimates the emotion using an emotion estimation algorithm. Additionally, when the user is relaxed, the evaluation unit can perform basic risk evaluation. For example, the evaluation unit records the user's voice and estimates the emotion using voice analysis technology. Furthermore, when the user is fatigued, the evaluation unit can prioritize evaluation of risks related to fatigue. For example, the evaluation unit collects the user's biometric data (such as heart rate and skin conductance) with sensors and estimates the emotion using an emotion estimation algorithm. In this way, by adjusting the criteria for risk evaluation according to the user's emotion, the evaluation unit enables more appropriate risk evaluation. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the evaluation unit may be performed using generative AI or without using generative AI. For example, the evaluation unit may input user image data captured by a camera into generative AI, which then estimates the emotion. Specifically, the evaluation unit is equipped with multiple high-resolution cameras, a microphone array, and biometric sensors (such as heart rate, skin conductance, skin temperature, etc.), and simultaneously acquires facial expression images (e.g., 30 frames×224×224×3), audio waveforms (5 seconds of audio sampled at 16 kHz), and biometric signal time series (5 seconds of heart rate and skin conductance samples). The evaluation unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82, relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the evaluation unit applies risk evaluation criteria adjustment logic (e.g., prioritize stress-related risks if stress intensity is 0.7 or higher, perform basic risk evaluation if relaxed, prioritize fatigue-related risks if fatigued) to automatically adjust selection of risk evaluation target data, evaluation order, and evaluation granularity. For example, when stress is high, risks directly related to stress such as heart rate variability, skin conductance, facial expression changes, and conversation content are prioritized for evaluation; when relaxed, basic health risks such as posture during walking and meal content are evaluated. These processes, unlike conventional subjective observation or uniform evaluation by humans, automate and optimize emotion estimation and risk evaluation criteria control in a high-dimensional feature space using multimodal AI, thereby fundamentally improving computer technology by enhancing risk evaluation accuracy, reducing user burden, improving data storage efficiency, and enabling early identification of stress factors. Furthermore, the evaluation unit achieves flexible and highly accurate risk evaluation according to user state by linking multiple AI modules such as a multimodal neural network for emotion estimation, evaluation criteria adjustment logic, and risk evaluation database management modules. Application fields include elderly care facilities, home health management, mental health monitoring, stress management support, and remote medical monitoring, and the technology is particularly effective in environments where emotional fluctuations are directly linked to health risks.

The evaluation unit can refer to past evaluation data during evaluation to appropriately select the evaluation algorithm. For example, the evaluation unit analyzes past evaluation data and selects the optimal evaluation algorithm. The evaluation unit may also adjust the evaluation method or algorithm parameters based on past evaluation data. Furthermore, the evaluation unit can optimize the algorithm by referring to past evaluation data to improve evaluation accuracy. Thus, by referring to past evaluation data, the evaluation unit can optimize the evaluation algorithm. Some or all of the above-described processing in the evaluation unit may be performed using generative AI or without using generative AI. For example, the evaluation unit may input past evaluation data into generative AI, which then optimizes the evaluation algorithm. Specifically, the evaluation unit is equipped with a database that manages time-series accumulated evaluation history data for each user (e.g., evaluation result scores for the past year, feature vectors of evaluated data, algorithm parameter history, etc.). The evaluation unit preprocesses these past evaluation data in a preprocessing unit for normalization, missing value completion, and time-series feature extraction (e.g., moving average, trend score, anomaly detection), and inputs the obtained feature vectors (e.g., 365×10-dimensional evaluation history array) into an evaluation algorithm optimization neural network (e.g., LSTM for time-series analysis and MLP for algorithm parameter control). This neural network learns patterns of past evaluation accuracy fluctuations, anomaly occurrence history, and correlations between algorithm parameters and evaluation accuracy, and outputs the optimal evaluation algorithm (e.g., tensor similarity calculation, dynamic time warping, feature space clustering), parameter sets (e.g., thresholds, weighting coefficients, evaluation target selection rules), and evaluation accuracy prediction scores. Examples of AI input include (1) time-series arrays of evaluation scores for the past year (365×3 dimensions), (2) algorithm parameter history (e.g., time-series of threshold 0.7, weight 0.5, etc.), and (3) anomaly detection history (e.g., list of false positive dates). AI output includes (1) recommended evaluation algorithm (e.g., “dynamic time warping”), (2) recommended parameter set (e.g., threshold 0.75, weight 0.6), and (3) evaluation accuracy prediction score (e.g., 0.92). The evaluation unit inputs these output values into evaluation control logic to automatically set optimized evaluation algorithms and parameters for each user and data type. For example, if there were many false positives in gait evaluation in the past, the algorithm is changed; if accuracy was high in meal evaluation, the current parameters are maintained. These processes, unlike conventional uniform algorithms or human experience-based evaluation settings, automate time-series evaluation data analysis and algorithm optimization using AI, thereby fundamentally improving computer technology by enhancing evaluation accuracy, anomaly detection rate, data storage efficiency, and reducing user burden. Furthermore, the evaluation unit achieves flexible and highly accurate evaluation according to each user's evaluation history and risk profile by linking multiple AI modules such as LSTM for time-series analysis, MLP for algorithm parameter control, and evaluation database management modules. Application fields include elderly care facilities, home health management, chronic disease monitoring, rehabilitation support, and remote medical monitoring, and the technology is particularly effective in environments where individual optimization is required.

The evaluation unit can adjust the frequency of evaluation during evaluation based on the user's health condition and lifestyle habits. For example, when the user's health condition changes, the evaluation unit adjusts the frequency of evaluation. The evaluation unit may adjust the frequency of evaluation based on health conditions such as blood pressure, heart rate, and body weight. Furthermore, when the user's lifestyle habits change, the evaluation unit can also adjust the frequency of evaluation. For example, the evaluation unit adjusts the frequency of evaluation based on meal patterns, exercise habits, and sleep habits. The evaluation unit can also optimize the frequency of evaluation based on the user's health condition and lifestyle habits. Thus, by adjusting the frequency of evaluation according to health condition and lifestyle habits, the evaluation unit enables appropriate evaluation. Some or all of the above-described processing in the evaluation unit may be performed using generative AI or without using generative AI. For example, the evaluation unit may input data on the user's health condition and lifestyle habits into generative AI, which then adjusts the frequency of evaluation. Specifically, the evaluation unit is equipped with a database that manages the user's health condition data (e.g., time-series data of blood pressure, heart rate, body weight, blood test values, etc.), lifestyle habit data (e.g., meal records, exercise records, sleep records, etc.), and past evaluation frequency and evaluation result history. The evaluation unit preprocesses these data in a preprocessing unit for normalization and feature extraction (e.g., weekly average health indicators, meal patterns, sleep rhythms, etc.), and inputs the obtained feature vectors (e.g., 7 days×10 health and lifestyle habit features) into an evaluation frequency optimization neural network (e.g., MLP for health features and LSTM for time-series). This neural network learns the correlation between health condition/lifestyle habit fluctuations and evaluation accuracy/health event occurrence, and outputs the optimal evaluation frequency (e.g., once per day in weeks with unstable health, once per week in stable weeks). Examples of AI input include (1) time-series arrays of blood pressure, heart rate, and body weight for one week (7×3 dimensions), (2) one week of meal, exercise, and sleep records (7×3 dimensions), and (3) past evaluation frequency and evaluation result history (e.g., daily evaluation, weekly evaluation, etc.). AI output includes (1) recommended evaluation frequency (e.g., once per day), and (2) recommended evaluation items (e.g., “exercise”, “sleep”). The evaluation unit inputs these output values into evaluation control logic to automatically adjust evaluation frequency and evaluation items according to health condition and lifestyle habits. For example, when health condition worsens, evaluation frequency is increased; when stable, evaluation frequency is decreased. These processes, unlike conventional uniform evaluation or subjective human judgment, automate evaluation and evaluation frequency optimization in a high-dimensional feature space of health and lifestyle habit data using AI, thereby fundamentally improving computer technology by enhancing evaluation accuracy, data storage efficiency, reducing user burden, and improving responsiveness to health fluctuations. Furthermore, the evaluation unit achieves flexible and highly accurate evaluation according to each user's health condition and lifestyle habits by linking multiple AI modules such as MLP for health feature extraction, LSTM for time-series analysis, and evaluation frequency control logic. Application fields include elderly care facilities, home health management, rehabilitation support, remote medical monitoring, and environments where health condition fluctuations affect health, and the technology is particularly effective in situations where health-adaptive monitoring is required.

The evaluation unit can estimate a user's emotion and adjust the display method of evaluation results based on the estimated emotion. For example, when the user is feeling stressed, the evaluation unit provides a simple and highly visible display method. For instance, the evaluation unit captures the user's facial expressions with a camera and estimates the emotion using an emotion estimation algorithm. Additionally, when the user is relaxed, the evaluation unit can provide a display method that includes detailed information. For example, the evaluation unit records the user's voice and estimates the emotion using voice analysis technology. Furthermore, when the user is fatigued, the evaluation unit can provide a display method that highlights key points. For example, the evaluation unit collects the user's biometric data (such as heart rate and skin conductance) with sensors and estimates the emotion using an emotion estimation algorithm. In this way, by adjusting the display method of evaluation results according to the user's emotion, the evaluation unit enables more appropriate display. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the evaluation unit may be performed using AI or without using AI. For example, the evaluation unit may input user image data captured by a camera into generative AI, which then estimates the emotion. Specifically, the evaluation unit is equipped with multiple high-resolution cameras, a microphone array, and biometric sensors (such as heart rate, skin conductance, skin temperature, etc.), and simultaneously acquires facial expression images (e.g., 30 frames×224×224 ×3), audio waveforms (5 seconds of audio sampled at 16 kHz), and biometric signal time series (5 seconds of heart rate and skin conductance samples). The evaluation unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82, relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the evaluation unit applies display method control logic (e.g., simple display if stress intensity is 0.7 or higher, detailed display if relaxed, key point display if fatigued) to automatically adjust the display content, display granularity, and display order of evaluation results. For example, when stress is high, only major health indicators are displayed in large font and color-coded; when relaxed, detailed graphs, time-series variations, and basis data are displayed. These processes, unlike conventional uniform display or subjective human judgment, automate and optimize emotion estimation and display control in a high-dimensional feature space using multimodal AI, thereby fundamentally improving computer technology by reducing user cognitive burden, improving information transmission efficiency, and enabling early identification of stress factors. Furthermore, the evaluation unit achieves flexible and highly accurate display according to user state by linking multiple AI modules such as a multimodal neural network for emotion estimation, display method control logic, and user interface management modules. Application fields include elderly care facilities, home health management, mental health monitoring, stress management support, and remote medical monitoring, and the technology is particularly effective in environments where emotional fluctuations are directly linked to health risks and information receptivity.

The evaluation unit can perform evaluation during evaluation based on the user's geographic distribution. For example, when the user is in a specific region, the evaluation unit evaluates data related to that region. For instance, the evaluation unit considers the user's geographic distribution and evaluates health information related to that region. Additionally, when the user is traveling, the evaluation unit can evaluate data related to the travel destination. For example, the evaluation unit considers the user's geographic distribution at the travel destination and evaluates health information related to travel. Furthermore, the evaluation unit can select the optimal evaluation method based on the user's geographic distribution. For example, the evaluation unit optimizes the algorithm based on the user's geographic distribution to improve evaluation accuracy. Thus, by performing evaluation based on geographic distribution, the evaluation unit enables more appropriate evaluation. Some or all of the above-described processing in the evaluation unit may be performed using generative AI or without using generative AI. For example, the evaluation unit may input the user's geographic distribution data into generative AI, which then performs the evaluation. Specifically, the evaluation unit acquires the user's geographic location information (e.g., latitude, longitude, altitude, indoor coordinates, location labels, etc.) in real time using GPS modules, Wi-Fi/Bluetooth beacons, indoor positioning sensors, etc. The evaluation unit preprocesses these location data in a preprocessing unit for normalization, geographic clustering, and location labeling (e.g., home, workplace, hospital, travel destination, etc.), and inputs the obtained feature vectors (e.g., current location label plus past 24-hour movement history vector) into a location-dependent evaluation optimization neural network (e.g., MLP for location features and LSTM for time-series movement history). This neural network learns the correlation between health risks, behavior patterns, and evaluation priority items for each location, and outputs a location-based evaluation priority list (e.g., prioritize physical condition changes, meal content, and movement amount at travel destinations; prioritize daily life indicators at home) and optimal evaluation algorithms (e.g., automatically adjust thresholds and weights for each location). Examples of AI input include (1) current location latitude, longitude, and location label, (2) past 24-hour movement history vector (24×3 dimensions), and (3) health event occurrence history for each location. AI output includes (1) recommended evaluation priority list (e.g., “Travel destination: physical condition change >meal content >movement amount”, “Home: sleep >meal >exercise”), (2) recommended evaluation algorithm (e.g., “dynamic time warping”), and (3) recommended parameter set (e.g., threshold 0.75, weight 0.6). The evaluation unit inputs these output values into evaluation control logic to automatically adjust selection of evaluation target data, evaluation order, evaluation algorithm, and parameters according to current location and movement status. For example, during travel, physical condition changes and meal content are evaluated with emphasis, while at home, daily life indicators are evaluated regularly. These processes, unlike conventional uniform evaluation or subjective human judgment, automate and optimize evaluation and evaluation optimization in a high-dimensional feature space of geographic location information using AI, thereby fundamentally improving computer technology by enhancing evaluation accuracy, anomaly detection rate, data storage efficiency, and reducing user burden. Furthermore, the evaluation unit achieves flexible and highly accurate evaluation according to the user's geographic location and behavior patterns by linking multiple AI modules such as MLP for location feature extraction, LSTM for movement history analysis, and evaluation priority control logic. Application fields include elderly care facilities, home health management, traveler health monitoring, remote medical monitoring, and environments where changes in living environment affect health, and the technology is particularly effective in situations where movement or environmental changes are directly linked to health risks and evaluation accuracy.

The evaluation unit can refer to related literature during evaluation to improve evaluation accuracy. For example, the evaluation unit refers to literature related to the user's health to improve evaluation accuracy. The evaluation unit may also refer to academic papers, medical guidelines, and specialized books to improve evaluation accuracy. Furthermore, the evaluation unit can refer to literature related to the user's lifestyle habits to improve evaluation accuracy. For example, the evaluation unit improves evaluation accuracy based on literature related to the user's lifestyle habits. Additionally, the evaluation unit can refer to literature related to the user's activity patterns to improve evaluation accuracy. For example, the evaluation unit improves evaluation accuracy based on literature related to the user's activity patterns. Thus, by referring to related literature, the evaluation unit can improve evaluation accuracy. Some or all of the above-described processing in the evaluation unit may be performed using generative AI or without using generative AI. For example, the evaluation unit may input the user's related literature data into generative AI, which then improves evaluation accuracy. Specifically, the evaluation unit is configured to refer to a literature database containing academic papers, medical guidelines, specialized books, and the latest research results related to the user's health condition, lifestyle habits, and activity patterns. The evaluation unit preprocesses these literature data in a preprocessing unit for text normalization, summarization, keyword extraction, and knowledge graph construction (e.g., extraction of relationships among disease names, symptoms, recommended countermeasures, and risk factors), and inputs the obtained knowledge vectors (e.g., 256-dimensional literature summary embeddings, risk factor relevance scores) into a literature-referenced evaluation optimization neural network (e.g., Transformer for text and an integrated layer for knowledge graph embeddings). This neural network learns the correlation between the user's health data, lifestyle habits, activity patterns, and literature knowledge, and outputs selection of evaluation algorithms, parameter adjustment, and generation of explanation texts for evaluation rationale (e.g., “This evaluation is based on XX guideline”). Examples of AI input include (1) literature summary vectors related to the user's health indicators, (2) knowledge graphs related to lifestyle habits and activity patterns, and (3) relevance scores between evaluation target data and literature risk factors. AI output includes (1) recommended evaluation algorithm (e.g., “guideline-compliant type”), (2) recommended parameter set (e.g., threshold 0.8, weight 0.7), and (3) evaluation rationale explanation text (e.g., “Emphasis on decreased walking speed based on XX paper”). The evaluation unit inputs these output values into evaluation control logic to automatically set evaluation algorithms, parameters, and rationale explanations based on literature knowledge. For example, indicators recommended in the latest medical guidelines are prioritized for evaluation, and when the risk of lifestyle-related diseases is high, thresholds from related literature are applied. These processes, unlike conventional experience-based or subjective human judgment, automate and optimize evaluation and evaluation optimization in a high-dimensional feature space of literature knowledge using AI, thereby fundamentally improving computer technology by enhancing evaluation accuracy, rationale explainability, user reliability, and data management efficiency. Furthermore, the evaluation unit achieves flexible and highly accurate evaluation according to each user's health condition, lifestyle habits, and activity patterns by linking multiple AI modules such as Transformer for text analysis, knowledge graph integration layers, and evaluation rationale explanation generation modules. Application fields include elderly care facilities, home health management, chronic disease monitoring, rehabilitation support, remote medical monitoring, and evidence-based medical support, and the technology is particularly effective in environments where rationale explainability and accuracy are emphasized.

The proposal unit can estimate a user's emotion and adjust the expression method of proposals based on the estimated emotion. For example, when the user is feeling stressed, the proposal unit provides simple and highly visible proposals. For instance, the proposal unit captures the user's facial expressions with a camera and estimates the emotion using an emotion estimation algorithm. Additionally, when the user is relaxed, the proposal unit can provide proposals that include detailed information. For example, the proposal unit records the user's voice and estimates the emotion using voice analysis technology. Furthermore, when the user is fatigued, the proposal unit can provide proposals that highlight key points. For example, the proposal unit collects the user's biometric data (such as heart rate and skin conductance) with sensors and estimates the emotion using an emotion estimation algorithm. In this way, by adjusting the expression method of proposals according to the user's emotion, the proposal unit enables more appropriate proposals. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the proposal unit may be performed using AI or without using AI. For example, the proposal unit may input user image data captured by a camera into generative AI, which then estimates the emotion. Specifically, the proposal unit is equipped with multiple high-resolution cameras, a microphone array, and biometric sensors (such as heart rate, skin conductance, skin temperature, etc.), and simultaneously acquires facial expression images (e.g., 30 frames×224×224×3), audio waveforms (5 seconds of audio sampled at 16 kHz), and biometric signal time series (5 seconds of heart rate and skin conductance samples). The proposal unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82,relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the proposal unit applies proposal expression control logic (e.g., simple expression if stress intensity is 0.7 or higher, detailed expression if relaxed, key point expression if fatigued) to automatically adjust proposal content, expression granularity, display order, and format (e.g., text summary, graph, illustration, etc.). For example, when stress is high, only major countermeasures are displayed in large font and color-coded; when relaxed, proposals including detailed rationale and options are presented. These processes, unlike conventional uniform proposals or subjective human judgment, automate and optimize emotion estimation and proposal expression control in a high-dimensional feature space using multimodal AI, thereby fundamentally improving computer technology by reducing user cognitive burden, improving proposal acceptance, and enabling early identification of stress factors. Furthermore, the proposal unit achieves flexible and highly accurate proposals according to user state by linking multiple AI modules such as a multimodal neural network for emotion estimation, proposal expression control logic, and user interface management modules. Application fields include elderly care facilities, home health management, mental health monitoring, stress management support, and remote medical monitoring, and the technology is particularly effective in environments where emotional fluctuations are directly linked to health risks and information receptivity.

The proposal unit can adjust the level of detail of proposals during proposal generation based on the importance of risks. For example, when the risk is high, the proposal unit provides detailed proposals. For instance, the proposal unit evaluates the importance of risks and proposes detailed countermeasures. Additionally, when the risk is moderate, the proposal unit can provide basic proposals. For example, the proposal unit evaluates the importance of risks and proposes basic countermeasures. Furthermore, when the risk is low, the proposal unit can provide concise proposals. For example, the proposal unit evaluates the importance of risks and proposes concise countermeasures. In this way, by adjusting the level of detail of proposals according to the importance of risks, the proposal unit enables more appropriate proposals. Some or all of the above-described processing in the proposal unit may be performed using generative AI or without using generative AI. For example, the proposal unit may input risk evaluation data into generative AI, which then adjusts the level of detail of proposals. Specifically, the proposal unit receives risk evaluation data output from the evaluation unit (e.g., risk scores, risk categories, risk occurrence probabilities, risk basis features, etc.), preprocesses these in a preprocessing unit for normalization and feature extraction (e.g., risk score distribution, importance clustering, etc.), and inputs the obtained feature vectors (e.g., 10-dimensional risk score, category label, occurrence probability, etc.) into a risk importance-based proposal generation neural network (e.g., MLP for risk features and proposal template selection layer). This neural network learns the optimal correspondence between risk importance and proposal detail/content, and outputs recommended proposal detail level (e.g., detailed, basic, concise), recommended proposal template (e.g., detailed countermeasure list, summary countermeasure, alert only, etc.), and recommended display format (e.g., text, graph, illustration, etc.). Examples of AI input include (1) risk score vector (e.g., risk scores for 10 items), (2) risk category label (e.g., “fall”, “malnutrition”, etc.), and (3) risk occurrence probability (e.g., 0.85). AI output includes (1) recommended proposal detail level (e.g., “detailed”), (2) recommended proposal template (e.g., “exercise therapy+dietary improvement+medical consultation”), and (3) recommended display format (e.g., “detailed text+graph”). The proposal unit inputs these output values into proposal generation logic to automatically adjust proposal content, detail level, and display method according to risk importance. For example, when the risk score is high, a detailed countermeasure list and rationale explanation are presented; when moderate, only basic countermeasures are presented; when low, only alerts or concise advice are displayed. These processes, unlike conventional uniform proposals or subjective human judgment, automate and optimize risk evaluation data analysis and proposal detail optimization in a high-dimensional feature space using AI, thereby fundamentally improving computer technology by enhancing proposal accuracy, reducing user burden, and improving responsiveness to risks. Furthermore, the proposal unit achieves flexible and highly accurate proposals according to each user's risk profile by linking multiple AI modules such as MLP for risk feature extraction, proposal template selection layer, and proposal generation logic. Application fields include elderly care facilities, home health management, chronic disease monitoring, rehabilitation support, and remote medical monitoring, and the technology is particularly effective in environments where individual optimization according to risk importance is required.

The proposal unit can apply different proposal algorithms during proposal generation according to the category of risks. For example, in the case of health risks, the proposal unit provides proposals related to health management. For instance, the proposal unit evaluates health risks and proposes countermeasures related to health management. Additionally, in the case of lifestyle risks, the proposal unit can provide proposals related to lifestyle improvement. For example, the proposal unit evaluates lifestyle risks and proposes countermeasures related to lifestyle improvement. Furthermore, in the case of environmental risks, the proposal unit can provide proposals related to environmental improvement. For example, the proposal unit evaluates environmental risks and proposes countermeasures related to environmental improvement. In this way, by applying different proposal algorithms according to the category of risks, the proposal unit enables more appropriate proposals. Some or all of the above-described processing in the proposal unit may be performed using generative AI or without using generative AI. For example, the proposal unit may input risk evaluation data into generative AI, which then applies proposal algorithms. Specifically, the proposal unit receives risk evaluation data output from the evaluation unit (e.g., risk category label, risk score, risk basis features, etc.), preprocesses these in a preprocessing unit for feature extraction and normalization by category (e.g., features for health risks, lifestyle risks, environmental risks, etc.), and inputs the obtained feature vectors (e.g., category-specific feature vectors) into a risk category-based proposal generation neural network (e.g., category branching layer, category-specific MLPs, proposal template selection layer). This neural network learns the optimal proposal algorithms, countermeasure content, and expression methods for each risk category, and outputs recommended proposal algorithms (e.g., health risk: guideline-compliant type, lifestyle risk: behavior change support type, environmental risk: environmental improvement recommendation type), recommended proposal templates (e.g., exercise therapy, dietary improvement, room temperature adjustment, etc.), and recommended display formats. Examples of AI input include (1) risk category label (e.g., “health”, “lifestyle”, “environment”), (2) category-specific feature vectors (e.g., 10 dimensions for health risk, 8 for lifestyle risk, etc.), and (3) risk score. AI output includes (1) recommended proposal algorithm (e.g., “guideline-compliant type”), (2) recommended proposal template (e.g., “exercise therapy+dietary improvement”), and (3) recommended display format (e.g., “detailed text+illustration”). The proposal unit inputs these output values into proposal generation logic to automatically adjust proposal algorithms, content, and display method for each risk category. For example, in the case of health risks, detailed medical countermeasures are presented; for lifestyle risks, proposals to promote behavior change are provided; for environmental risks, environmental adjustment measures are prioritized. These processes, unlike conventional uniform proposals or subjective human judgment, automate and optimize AI analysis and proposal algorithm optimization for each risk category, thereby fundamentally improving computer technology by enhancing proposal accuracy, reducing user burden, and improving responsiveness to risks. Furthermore, the proposal unit achieves flexible and highly accurate proposals according to each user's risk category by linking multiple AI modules such as category branching layers, category-specific MLPs, and proposal template selection layers. Application fields include elderly care facilities, home health management, chronic disease monitoring, rehabilitation support, remote medical monitoring, and environments where changes in living environment affect health, and the technology is particularly effective in situations where individual optimization according to risk category is required.

The proposal unit can estimate a user's emotion and adjust the length of proposals based on the estimated emotion. For example, when the user is feeling stressed, the proposal unit provides short and concise proposals that highlight key points. For instance, the proposal unit captures the user's facial expressions with a camera and estimates the emotion using an emotion estimation algorithm. Additionally, when the user is relaxed, the proposal unit can provide detailed proposals. For example, the proposal unit records the user's voice and estimates the emotion using voice analysis technology. Furthermore, when the user is fatigued, the proposal unit can provide concise proposals. For example, the proposal unit collects the user's biometric data (such as heart rate and skin conductance) with sensors and estimates the emotion using an emotion estimation algorithm. In this way, by adjusting the length of proposals according to the user's emotion, the proposal unit enables more appropriate proposals. Emotion estimation is realized, for example, by using an emotion engine or an emotion estimation function implemented with generative AI. Generative AI may include text generation AI (such as LLM) or multimodal generative AI, but is not limited to these examples. Some or all of the above-described processing in the proposal unit may be performed using AI or without using AI. For example, the proposal unit may input user image data captured by a camera into generative AI, which then estimates the emotion. Specifically, the proposal unit is equipped with multiple high-resolution cameras, a microphone array, and biometric sensors (such as heart rate, skin conductance, skin temperature, etc.), and simultaneously acquires facial expression images (e.g., 30frames×224×224×3), audio waveforms (5 seconds of audio sampled at 16 kHz), and biometric signal time series (5 seconds of heart rate and skin conductance samples). The proposal unit preprocesses these multimodal data in a preprocessing unit for noise removal, normalization, and feature extraction (e.g., facial expression feature vectors, audio spectral features, heart rate variability indices), and inputs the obtained feature vectors (e.g., 128 dimensions for facial expression, 64 for audio, 32 for biometric data) into a multimodal emotion estimation neural network (e.g., an architecture integrating CNN for images, RNN for audio, and MLP for biometric data). This emotion estimation neural network integrates features from each modality using a self-attention mechanism and outputs emotion state labels (e.g., “stress”, “relaxation”, “fatigue”, etc.) and emotion intensity scores (e.g., stress 0.82, relaxation 0.15). Examples of AI input include (1) facial image tensors (30×224×224×3), (2) audio waveforms (5 seconds of PCM data), and (3) time series arrays of heart rate and skin conductance (5 seconds of sample values). AI output includes (1) emotion state labels (e.g., “stress”), (2) emotion intensity scores (e.g., stress 0.82), and (3) estimated basis features (e.g., contraction degree of the depressor anguli oris muscle, audio pitch variation, heart rate variability index). Based on these output values, the proposal unit applies proposal length control logic (e.g., short key-point proposals if stress intensity is 0.7 or higher, detailed proposals if relaxed, concise proposals if fatigued) to automatically adjust proposal content, length, and display method. For example, when stress is high, only key points are presented in short sentences; when relaxed, detailed rationale and options are presented in long proposals. These processes, unlike conventional uniform proposals or subjective human judgment, automate and optimize emotion estimation and proposal length control in a high-dimensional feature space using multimodal AI, thereby fundamentally improving computer technology by reducing user cognitive burden, improving proposal acceptance, and enabling early identification of stress factors. Furthermore, the proposal unit achieves flexible and highly accurate proposals according to user state by linking multiple AI modules such as a multimodal neural network for emotion estimation, proposal length control logic, and user interface management modules. Application fields include elderly care facilities, home health management, mental health monitoring, stress management support, and remote medical monitoring, and the technology is particularly effective in environments where emotional fluctuations are directly linked to health risks and information receptivity.

The proposal unit can determine the priority of proposals during proposal generation based on the occurrence timing of risks. For example, when a risk is expected to occur in the near future, the proposal unit prioritizes proposals. For instance, the proposal unit evaluates the occurrence timing of risks and proposes countermeasures with priority. Additionally, when a risk is expected to occur in the medium term, the proposal unit can provide basic proposals. For example, the proposal unit evaluates the occurrence timing of risks and proposes basic countermeasures. Furthermore, when a risk is expected to occur in the long term, the proposal unit can provide concise proposals. For example, the proposal unit evaluates the occurrence timing of risks and proposes concise countermeasures. In this way, by determining the priority of proposals according to the occurrence timing of risks, the proposal unit enables more appropriate proposals. Some or all of the above-described processing in the proposal unit may be performed using generative AI or without using generative AI. For example, the proposal unit may input risk evaluation data into generative AI, which then determines the priority of proposals. Specifically, the proposal unit receives risk evaluation data output from the evaluation unit (e.g., risk occurrence timing label, risk score, risk basis features, etc.), preprocesses these in a preprocessing unit for feature extraction and normalization by timing (e.g., labeling as near-term, medium-term, long-term, occurrence probability estimation, etc.), and inputs the obtained feature vectors (e.g., risk occurrence timing vector, score, etc.) into a risk occurrence timing-based proposal priority determination neural network (e.g., timing branching layer, priority score generation MLP). This neural network learns the optimal correspondence between risk occurrence timing and proposal priority/content, and outputs recommended proposal priority list (e.g., near-term risk prioritized, medium-term risk next, long-term risk deferred), recommended proposal template (e.g., immediate countermeasures, planned countermeasures, alerts, etc.), and recommended display format. Examples of AI input include (1) risk occurrence timing label (e.g., “near-term”, “medium-term”, “long-term”), (2) risk score, and (3) occurrence probability. AI output includes (1) recommended proposal priority list (e.g., “near-term risk: highest priority”), (2) recommended proposal template (e.g., “immediate countermeasures”), and (3) recommended display format (e.g., “emphasized display”). The proposal unit inputs these output values into proposal generation logic to automatically adjust proposal content, priority, and display method according to risk occurrence timing. For example, immediate countermeasures are prioritized for near-term risks, planned countermeasures are presented for medium-term risks, and alerts or concise advice are displayed for long-term risks. These processes, unlike conventional uniform proposals or subjective human judgment, automate and optimize AI analysis and proposal priority optimization for each risk occurrence timing, thereby fundamentally improving computer technology by enhancing proposal accuracy, reducing user burden, and improving responsiveness to risks. Furthermore, the proposal unit achieves flexible and highly accurate proposals according to each user's risk occurrence timing by linking multiple AI modules such as timing branching layers, priority score generation MLP, and proposal generation logic. Application fields include elderly care facilities, home health management, chronic disease monitoring, rehabilitation support, remote medical monitoring, and environments where individual optimization according to risk occurrence timing is required.

The proposal unit can adjust the order of proposals during proposal generation based on the relevance of risks. For example, when the risk is high, the proposal unit presents proposals first. For instance, the proposal unit evaluates the relevance of risks and proposes countermeasures first. Additionally, when the risk is moderate, the proposal unit can present proposals next. For example, the proposal unit evaluates the relevance of risks and proposes countermeasures next. Furthermore, when the risk is low, the proposal unit can present proposals last. For example, the proposal unit evaluates the relevance of risks and proposes countermeasures last. In this way, by adjusting the order of proposals according to the relevance of risks, the proposal unit enables more appropriate proposals. Some or all of the above-described processing in the proposal unit may be performed using generative AI or without using generative AI. For example, the proposal unit may input risk evaluation data into generative AI, which then adjusts the order of proposals. Specifically, the proposal unit receives multiple risk evaluation data output from the evaluation unit (e.g., risk scores, risk categories, risk relevance matrices, etc.), preprocesses these in a preprocessing unit for relevance score calculation, clustering, and normalization (e.g., causal relationship estimation between risks, weighting, etc.), and inputs the obtained feature vectors (e.g., risk relevance matrix, score vector, etc.) into a risk relevance-based proposal order optimization neural network (e.g., relevance graph embedding layer, order determination MLP). This neural network learns the optimal correspondence between risk relevance and proposal order/content, and outputs recommended proposal order list (e.g., high relevance risks prioritized, medium relevance risks next, low relevance risks deferred), recommended proposal template (e.g., composite countermeasures, individual countermeasures, etc.), and recommended display format (e.g., grouped display). Examples of AI input include (1) risk relevance matrix (e.g., 5×5 dimensions), (2) risk score vector, and (3) risk category label. AI output includes (1) recommended proposal order list (e.g., “high relevance risk: first”, “medium relevance risk: next”, “low relevance risk: last”), (2) recommended proposal template (e.g., “composite countermeasures”), and (3) recommended display format (e.g., “grouped display”). The proposal unit inputs these output values into proposal generation logic to automatically adjust proposal content, order, and display method according to risk relevance. For example, when multiple risks are mutually related, composite countermeasures are presented first, and individual countermeasures for less related risks are presented later. These processes, unlike conventional uniform proposals or subjective human judgment, automate and optimize risk relevance analysis and proposal order optimization in a high-dimensional feature space using AI, thereby fundamentally improving computer technology by enhancing proposal accuracy, reducing user burden, and improving responsiveness to risks. Furthermore, the proposal unit achieves flexible and highly accurate proposals according to each user's risk relevance by linking multiple AI modules such as relevance graph embedding layers, order determination MLP, and proposal generation logic. Application fields include elderly care facilities, home health management, chronic disease monitoring, rehabilitation support, remote medical monitoring, and environments where simultaneous management of multiple risks and relevance emphasis are required.

The system according to the embodiment is not limited to the examples described above, and various modifications are possible, for example, as follows.

The camera system may further include a voice recognition unit. The voice recognition unit can analyze the user's voice and estimate the health condition from daily conversations or soliloquy. For example, the voice recognition unit can analyze the user's tone, speaking speed, and content to detect signs of stress or fatigue. Additionally, when the user utters specific keywords (for example, pain or discomfort), the voice recognition unit can record such information and provide it to the analysis unit. In this way, the camera system enables more comprehensive health condition monitoring by utilizing the user's voice data.

The camera system may further include an environmental sensor unit. The environmental sensor unit can monitor the user's living environment (for example, room temperature, humidity, illuminance, etc.) and detect factors that affect health conditions. For example, the environmental sensor unit can issue a warning when the room temperature is too high or the humidity is too low. In addition, the environmental sensor unit can propose adjustments to lighting when the illuminance is inappropriate. Thus, the camera system enables health management that takes the user's living environment into consideration.

The camera system may further include an exercise analysis unit. The exercise analysis unit can analyze the user's exercise patterns in detail and detect lack of exercise or excessive exercise. For example, the exercise analysis unit can record the user's number of steps, exercise time, and exercise intensity, and propose an appropriate amount of exercise. Furthermore, the exercise analysis unit can analyze the user's exercise form and encourage exercise with correct form. Thus, the camera system can support the user's exercise habits and contribute to maintaining health.

The camera system may further include a nutrition management unit. The nutrition management unit can analyze the user's dietary content and evaluate nutritional balance. For example, the nutrition management unit can record the ingredients and amounts consumed by the user and detect excesses or deficiencies of nutrients. In addition, the nutrition management unit can propose meals tailored to the user's health condition. Thus, the camera system can support the user's dietary habits and contribute to maintaining health.

The camera system may further include a sleep analysis unit. The sleep analysis unit can analyze the user's sleep patterns and evaluate sleep quality. For example, the sleep analysis unit can record the number of times the user turns over and movements during sleep, and analyze the depth of sleep and frequency of interruptions. Furthermore, the sleep analysis unit can monitor the user's sleep environment (for example, room temperature and lighting) and propose an appropriate sleep environment. Thus, the camera system can improve the user's sleep quality and contribute to maintaining health.

The camera system may further include an emotion estimation unit. The emotion estimation unit can analyze the user's facial expressions, voice, and movements to estimate emotional states. For example, the emotion estimation unit can capture the user's facial expressions with a camera and estimate emotions using an emotion estimation algorithm. In addition, the emotion estimation unit can record the user's voice and estimate emotions using voice analysis technology. Thus, the camera system can grasp the user's emotional state and detect signs of stress or anxiety at an early stage.

The camera system may further include a reminder function. The reminder function can issue reminders at appropriate times based on the user's emotional state. For example, when the user is feeling stressed, the reminder function can propose a break for relaxation. In addition, when the user is relaxed, the reminder function can propose the timing for exercise or meals. Thus, the camera system can provide reminders according to the user's emotional state and support health management.

The camera system may further include a communication support unit. The communication support unit can propose appropriate communication methods based on the user's emotional state. For example, when the user is feeling stressed, the communication support unit can propose topics or methods that help the user relax. In addition, when the user is relaxed, the communication support unit can make proposals to encourage active communication. Thus, the camera system can provide communication support according to the user's emotional state and contribute to maintaining social health.

The camera system may further include a mental health support unit. The mental health support unit can analyze the user's emotional state and provide advice related to mental health. For example, the mental health support unit can analyze the user's facial expressions and voice to detect signs of stress or anxiety. In addition, the mental health support unit can propose relaxation methods or counseling tailored to the user's emotional state. Thus, the camera system can support the user's mental health and contribute to overall health maintenance.

The camera system may further include an emotion feedback function. The emotion feedback function can provide real-time feedback on the user's emotional state and promote self-awareness. For example, the emotion feedback function can analyze the user's facial expressions and voice and display the emotional state. In addition, when the user is feeling stressed, the emotion feedback function can identify the cause and propose countermeasures. Thus, the camera system can visualize the user's emotional state and support self-management.

The following is a brief description of the processing flow of Example of the Embodiment.
Step 1: The recording unit records movements in a healthy state. For example, the recording unit can record posture or speed during walking, or eating habits. The recording unit may, for example, capture posture during walking with a camera and analyze the data using AI. In addition, the recording unit may capture eating habits with a camera and analyze the data using AI.
Step 2: The comparison unit records daily movements. For example, the comparison unit can record daily walking or eating conditions. The comparison unit may, for example, capture daily walking with a camera and analyze the data using AI. In addition, the comparison unit may capture daily eating conditions with a camera and analyze the data using AI.
Step 3: The analysis unit analyzes data recorded by the recording unit and the comparison unit. For example, the analysis unit compares data in a healthy state with daily data and detects fluctuations in movement. The analysis unit may, for example, compare walking speed in a healthy state with daily walking speed and detect fluctuations. In addition, the analysis unit may compare the amount of food intake in a healthy state with daily food intake and detect fluctuations.
Step 4: The evaluation unit evaluates risks based on data analyzed by the analysis unit. For example, the evaluation unit analyzes fluctuations in movement and evaluates potential risks. The evaluation unit may, for example, evaluate a decrease in walking speed as a risk. In addition, the evaluation unit may evaluate a decrease in food intake as a risk.
Step 5: The proposal unit proposes countermeasures based on risks evaluated by the evaluation unit. For example, when a high risk is determined, the proposal unit recommends preventive healthcare or supplements. The proposal unit may, for example, propose exercise therapy when walking speed decreases. In addition, the proposal unit may propose nutritional supplements when food intake decreases.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT® (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned recording unit, comparison unit, analysis unit, evaluation unit, and proposal unit is implemented, for example, by at least one of a smart device 14 and a data processing apparatus 12. For example, the recording unit captures movements in a healthy state of elderly people using a camera 42 of the smart device 14, and the captured data is analyzed by a specific processing unit 290 of the data processing apparatus 12. The comparison unit captures daily movements using the camera 42 of the smart device 14, and the captured data is analyzed by the specific processing unit 290 of the data processing apparatus 12. The analysis unit is implemented by the specific processing unit 290 of the data processing apparatus 12 and analyzes data recorded by the recording unit and the comparison unit. The evaluation unit is implemented by the specific processing unit 290 of the data processing apparatus 12 and evaluates risks based on the analyzed data. The proposal unit is implemented by the specific processing unit 290 of the data processing apparatus 12 and proposes countermeasures based on the evaluated risks. The correspondence between each unit and the device or control unit is not limited to the examples described above and various modifications are possible.

### Second Embodiment

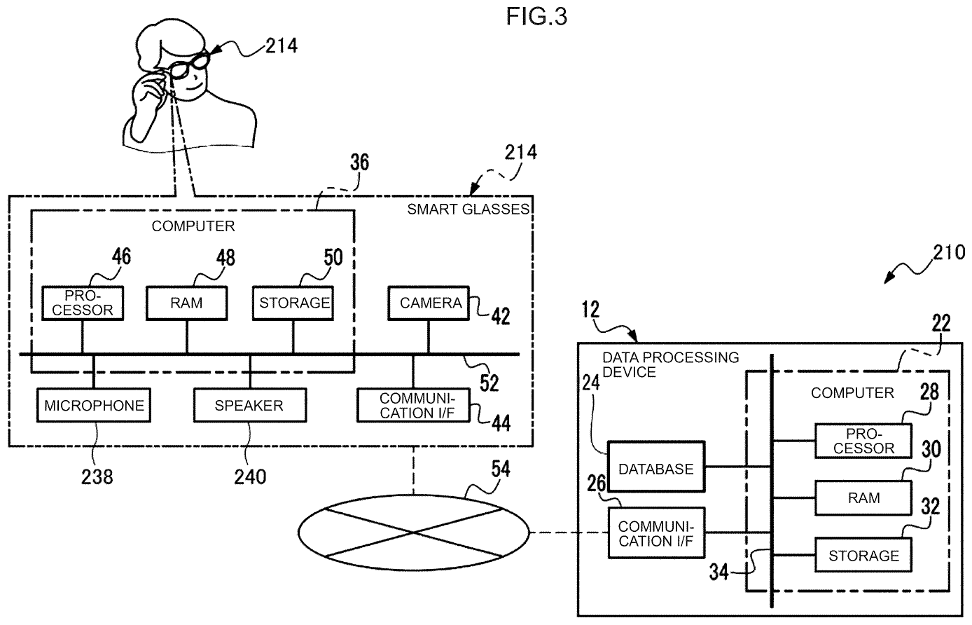

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

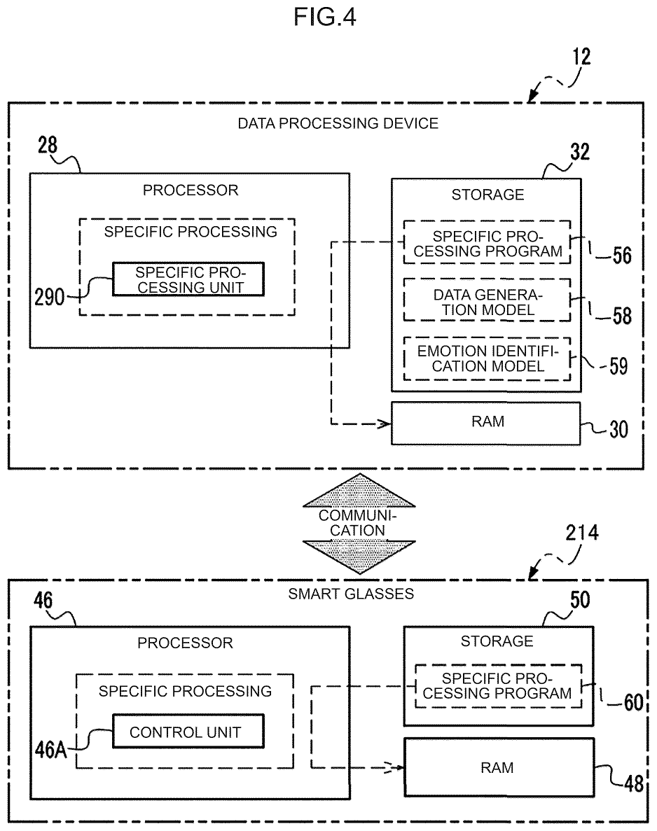

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned recording unit, comparison unit, analysis unit, evaluation unit, and proposal unit is implemented, for example, by at least one of smart glasses 214 and a data processing apparatus 12. For example, the recording unit captures movements in a healthy state of elderly people using a camera 42 of the smart glasses 214, and the captured data is analyzed by a specific processing unit 290 of the data processing apparatus 12. The comparison unit captures daily movements using the camera 42 of the smart glasses 214, and the captured data is analyzed by the specific processing unit 290 of the data processing apparatus 12. The analysis unit is implemented by the specific processing unit 290 of the data processing apparatus 12 and analyzes data recorded by the recording unit and the comparison unit. The evaluation unit is implemented by the specific processing unit 290 of the data processing apparatus 12 and evaluates risks based on the analyzed data. The proposal unit is implemented by the specific processing unit 290 of the data processing apparatus 12 and proposes countermeasures based on the evaluated risks. The correspondence between each unit and the device or control unit is not limited to the examples described above and various modifications are possible.

### Third Embodiment

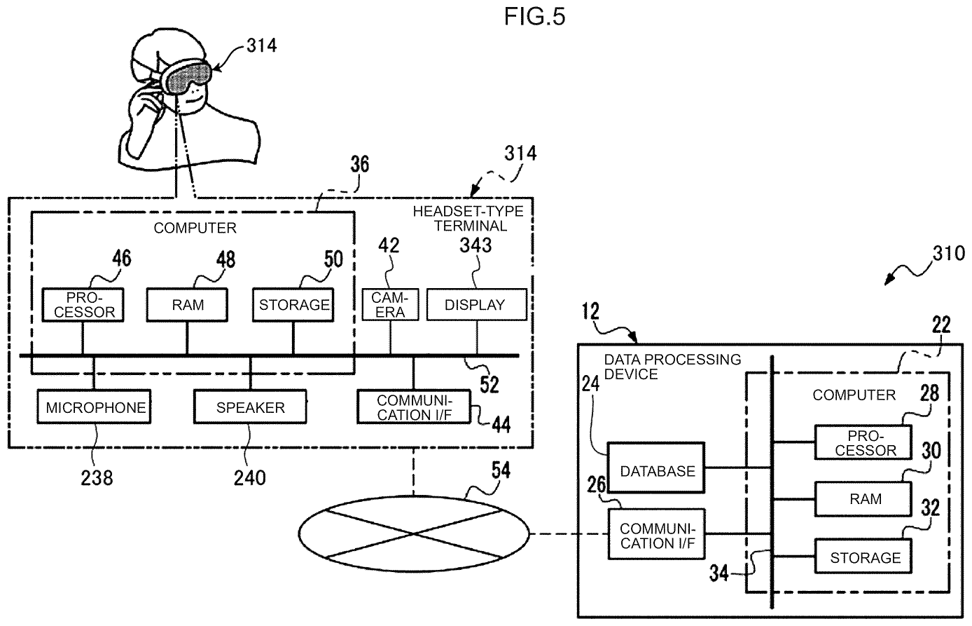

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

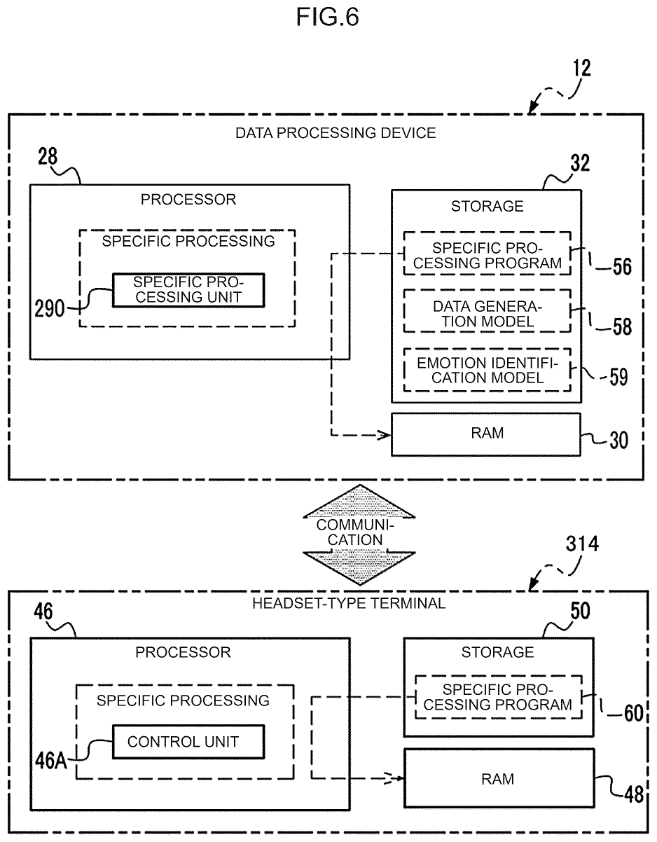

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned recording unit, comparison unit, analysis unit, evaluation unit, and proposal unit is implemented, for example, by at least one of a headset-type terminal 314 and a data processing apparatus 12. For example, the recording unit captures movements in a healthy state of elderly people using a camera 42 of the headset-type terminal 314, and the captured data is analyzed by a specific processing unit 290 of the data processing apparatus 12. The comparison unit captures daily movements using the camera 42 of the headset-type terminal 314, and the captured data is analyzed by the specific processing unit 290 of the data processing apparatus 12. The analysis unit is implemented by the specific processing unit 290 of the data processing apparatus 12 and analyzes data recorded by the recording unit and the comparison unit. The evaluation unit is implemented by the specific processing unit 290 of the data processing apparatus 12 and evaluates risks based on the analyzed data. The proposal unit is implemented by the specific processing unit 290 of the data processing apparatus 12 and proposes countermeasures based on the evaluated risks. The correspondence between each unit and the device or control unit is not limited to the examples described above and various modifications are possible.

### Fourth Embodiment

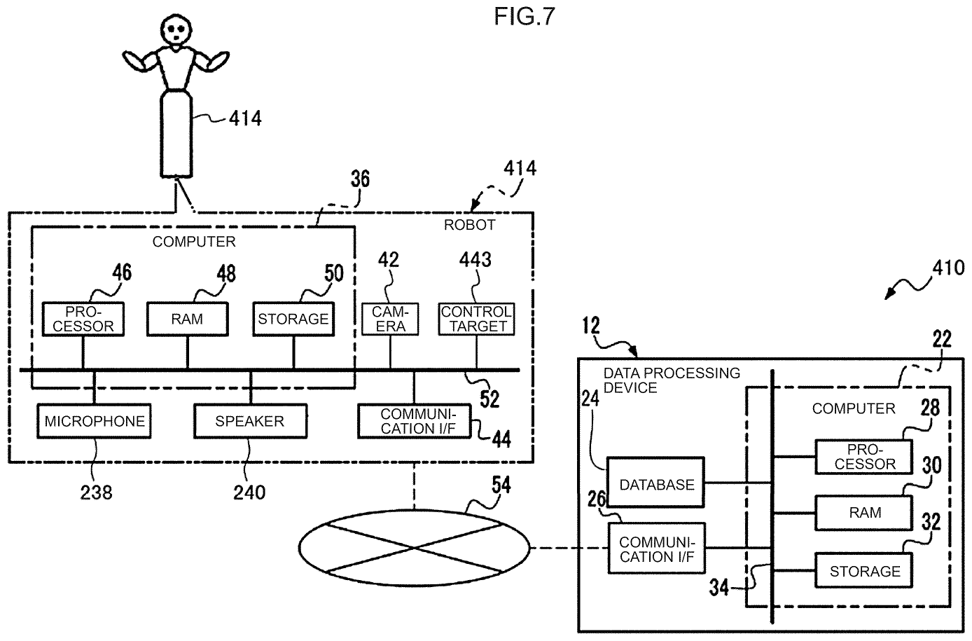

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

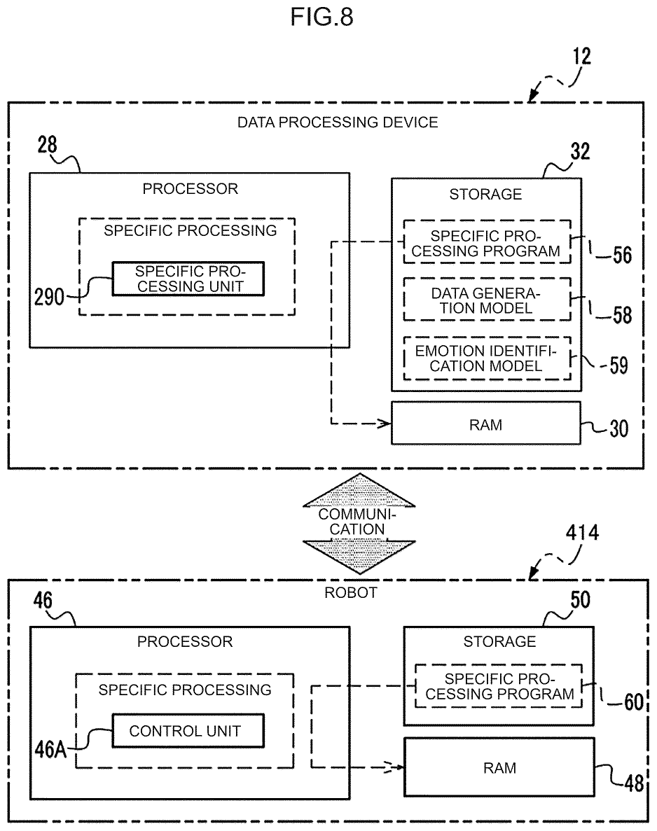

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned recording unit, comparison unit, analysis unit, evaluation unit, and proposal unit is implemented, for example, by at least one of a robot 414 and a data processing apparatus 12. For example, the recording unit captures movements in a healthy state of elderly people using a camera 42 of the robot 414, and the captured data is analyzed by a specific processing unit 290 of the data processing apparatus 12. The comparison unit captures daily movements using the camera 42 of the robot 414, and the captured data is analyzed by the specific processing unit 290 of the data processing apparatus 12. The analysis unit is implemented by the specific processing unit 290 of the data processing apparatus 12 and analyzes data recorded by the recording unit and the comparison unit. The evaluation unit is implemented by the specific processing unit 290 of the data processing apparatus 12 and evaluates risks based on the analyzed data. The proposal unit is implemented by the specific processing unit 290 of the data processing apparatus 12 and proposes countermeasures based on the evaluated risks. The correspondence between each unit and the device or control unit is not limited to the examples described above and various modifications are possible.

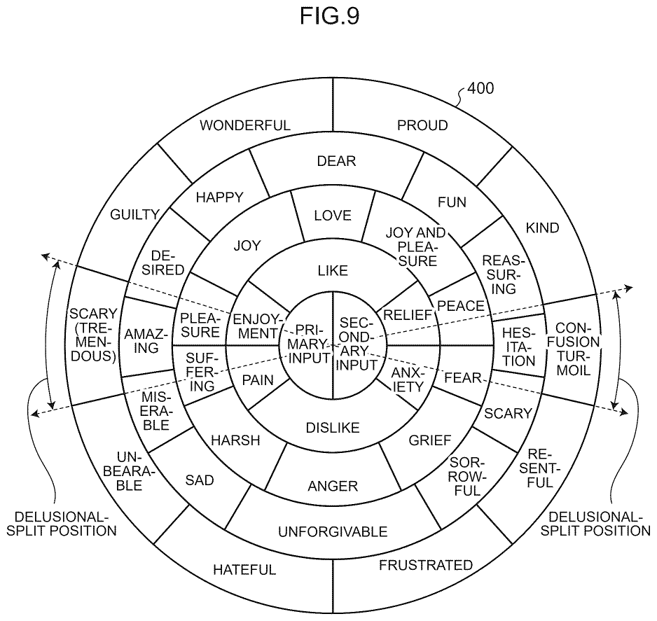

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

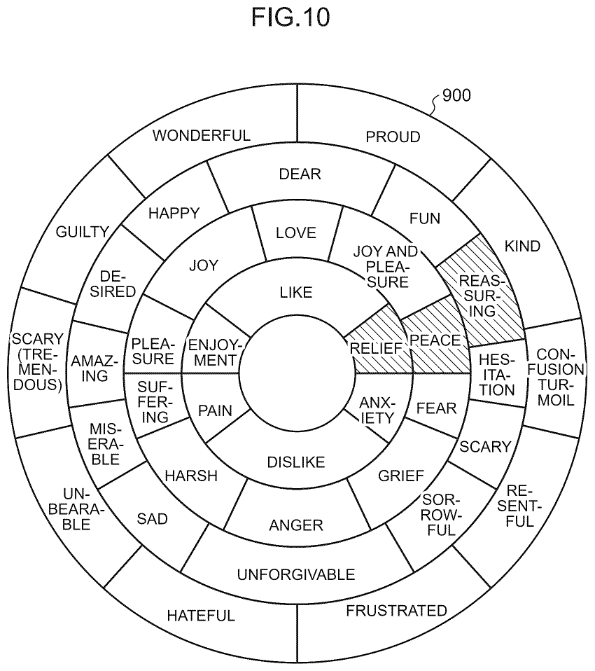

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a recording unit configured to record movements in a healthy state; a comparison unit configured to record daily movements; an analysis unit configured to analyze data recorded by the recording unit and the comparison unit; an evaluation unit configured to evaluate risks based on data analyzed by the analysis unit; and a proposal unit configured to propose countermeasures based on risks evaluated by the evaluation unit.
(Supplementary Note 2) The system according to Supplementary Note 1, wherein the recording unit is configured to record posture or speed during walking, or eating habits.
(Supplementary Note 3) The system according to Supplementary Note 1, wherein the comparison unit is configured to record daily walking or eating conditions.
(Supplementary Note 4) The system according to Supplementary Note 1, wherein the analysis unit is configured to compare data in a healthy state with daily data and detect fluctuations in movement.
(Supplementary Note 5) The system according to Supplementary Note 1, wherein the evaluation unit is configured to analyze fluctuations in movement and evaluate potential risks.
(Supplementary Note 6) The system according to Supplementary Note 1, wherein the proposal unit is configured to recommend preventive healthcare or supplements when a high risk is determined.
(Supplementary Note 7) The system according to Supplementary Note 1, wherein the recording unit is configured to estimate a user's emotion and adjust the level of detail of the data to be recorded based on the estimated emotion.
(Supplementary Note 8) The system according to Supplementary Note 1, wherein the recording unit is configured to select an appropriate recording method by referring to the user's past health data at the time of recording.
(Supplementary Note 9) The system according to Supplementary Note 1, wherein the recording unit is configured to adjust the recording frequency based on the user's living environment or activity level at the time of recording.
(Supplementary Note 10) The system according to Supplementary Note 1, wherein the recording unit is configured to estimate a user's emotion and determine the priority of data to be recorded based on the estimated emotion.
(Supplementary Note 11) The system according to Supplementary Note 1, wherein the recording unit is configured to preferentially record highly relevant data based on the user's geographic location information at the time of recording.
(Supplementary Note 12) The system according to Supplementary Note 1, wherein the recording unit is configured to analyze the user's social media activity and record relevant data at the time of recording.
(Supplementary Note 13) The system according to Supplementary Note 1, wherein the comparison unit is configured to estimate a user's emotion and adjust the comparison criteria based on the estimated emotion.
(Supplementary Note 14) The system according to Supplementary Note 1, wherein the comparison unit is configured to refer to past comparison data and appropriately select a comparison algorithm at the time of comparison.
(Supplementary Note 15) The system according to Supplementary Note 1, wherein the comparison unit is configured to adjust the comparison frequency based on the user's lifestyle or activity pattern at the time of comparison.
(Supplementary Note 16) The system according to Supplementary Note 1, wherein the comparison unit is configured to estimate a user's emotion and adjust the display method of comparison results based on the estimated emotion.
(Supplementary Note 17) The system according to Supplementary Note 1, wherein the comparison unit is configured to perform comparison based on the user's geographic distribution at the time of comparison.
(Supplementary Note 18) The system according to Supplementary Note 1, wherein the comparison unit is configured to refer to the user's related literature and improve the accuracy of comparison at the time of comparison.
(Supplementary Note 19) The system according to Supplementary Note 1, wherein the analysis unit is configured to estimate a user's emotion and adjust the level of detail of analysis based on the estimated emotion.
(Supplementary Note 20) The system according to Supplementary Note 1, wherein the analysis unit is configured to refer to past analysis data and appropriately select an analysis algorithm at the time of analysis.
(Supplementary Note 21) The system according to Supplementary Note 1, wherein the analysis unit is configured to adjust the analysis frequency based on the user's health condition or lifestyle at the time of analysis.
(Supplementary Note 22) The system according to Supplementary Note 1, wherein the analysis unit is configured to estimate a user's emotion and adjust the display method of analysis results based on the estimated emotion.
(Supplementary Note 23) The system according to Supplementary Note 1, wherein the analysis unit is configured to perform analysis based on the user's geographic distribution at the time of analysis.
(Supplementary Note 24) The system according to Supplementary Note 1, wherein the analysis unit is configured to refer to the user's related literature and improve the accuracy of analysis at the time of analysis.
(Supplementary Note 25) The system according to Supplementary Note 1, wherein the evaluation unit is configured to estimate a user's emotion and adjust the criteria for risk evaluation based on the estimated emotion.
(Supplementary Note 26) The system according to Supplementary Note 1, wherein the evaluation unit is configured to refer to past evaluation data and appropriately select an evaluation algorithm at the time of evaluation.
(Supplementary Note 27) The system according to Supplementary Note 1, wherein the evaluation unit is configured to adjust the evaluation frequency based on the user's health condition or lifestyle at the time of evaluation.
(Supplementary Note 28) The system according to Supplementary Note 1, wherein the evaluation unit is configured to estimate a user's emotion and adjust the display method of evaluation results based on the estimated emotion.
(Supplementary Note 29) The system according to Supplementary Note 1, wherein the evaluation unit is configured to perform evaluation based on the user's geographic distribution at the time of evaluation.
(Supplementary Note 30) The system according to Supplementary Note 1, wherein the evaluation unit is configured to refer to the user's related literature and improve the accuracy of evaluation at the time of evaluation.
(Supplementary Note 31) The system according to Supplementary Note 1, wherein the proposal unit is configured to estimate a user's emotion and adjust the expression method of proposals based on the estimated emotion.
(Supplementary Note 32) The system according to Supplementary Note 1, wherein the proposal unit is configured to adjust the level of detail of proposals based on the importance of risks at the time of proposal.
(Supplementary Note 33) The system according to Supplementary Note 1, wherein the proposal unit is configured to apply different proposal algorithms according to the category of risks at the time of proposal.
(Supplementary Note 34) The system according to Supplementary Note 1, wherein the proposal unit is configured to estimate a user's emotion and adjust the length of proposals based on the estimated emotion.
(Supplementary Note 35) The system according to Supplementary Note 1, wherein the proposal unit is configured to determine the priority of proposals based on the occurrence timing of risks at the time of proposal.
(Supplementary Note 36) The system according to Supplementary Note 1, wherein the proposal unit is configured to adjust the order of proposals based on the relevance of risks at the time of proposal.

What is claimed is:

1. A system comprising:
circuitry configured to:
acquire, via a communication interface coupled to a packet-switched network, first sensor data captured by an image sensor of a client terminal during a first time period and second sensor data captured by the image sensor of the client terminal during a second time period subsequent to the first time period;
extract, by inputting the first sensor data into a neural network comprising a Transformer encoder-decoder structure with a self-attention mechanism, a first feature vector representing a reference state;
extract, by inputting the second sensor data into the neural network, a second feature vector representing a current state;
compute a difference vector between the first feature vector and the second feature vector in a multidimensional feature space and generate an anomaly score based on the difference vector;
generate, by inputting the anomaly score and a classification label derived from the difference vector into a data generation model, response data comprising a recommended action; and
transmit the response data to the client terminal via the communication interface.

2. The system according to claim 1, wherein the first sensor data comprises time-series image tensors representing skeletal coordinate sequences extracted by a pose estimation algorithm, and wherein the first feature vector comprises a time-series array of joint coordinates.

3. The system according to claim 1, wherein the second sensor data comprises time-series image tensors representing motion patterns during at least one of locomotion or consumption activity, and wherein the second feature vector comprises a motion classification label generated by the neural network.

4. The system according to claim 1, wherein the circuitry is further configured to compute the anomaly score by calculating at least one of a cosine similarity or a Euclidean distance between the first feature vector and the second feature vector in the multidimensional feature space.

5. The system according to claim 1, wherein the classification label comprises at least one of a fluctuation type label indicating a category of detected change or a probability distribution across a plurality of fluctuation categories.

6. The system according to claim 1, wherein the data generation model comprises a large language model configured to generate the response data as a personalized text output based on the anomaly score, the classification label, and historical user data stored in a database.

7. The system according to claim 1, wherein the circuitry is further configured to estimate an emotion of a user by inputting multimodal sensor data received from the client terminal into an emotion identification model, and to adjust a granularity of the first sensor data or the second sensor data based on the estimated emotion, such that when the estimated emotion indicates a high-intensity state, the circuitry acquires the sensor data at a higher sampling frequency, and when the estimated emotion indicates a low-intensity state, the circuitry acquires the sensor data at a lower sampling frequency.

8. The system according to claim 1, wherein the circuitry is further configured to retrieve, from a database, past feature vectors associated with the client terminal and to select an extraction algorithm for extracting the first feature vector or the second feature vector based on the past feature vectors.

9. The system according to claim 1, wherein the circuitry is further configured to adjust a frequency of acquiring the second sensor data based on at least one of an activity level or an environmental parameter associated with the client terminal, such that the frequency increases when the activity level exceeds a threshold and decreases when the activity level falls below the threshold.

10. The system according to claim 1, wherein the circuitry is further configured to estimate an emotion of a user by inputting multimodal sensor data into an emotion identification model, and to determine a priority of elements within the first sensor data or the second sensor data based on the estimated emotion, such that when the estimated emotion indicates stress, the circuitry prioritizes sensor data associated with a stress-related attribute.

11. The system according to claim 1, wherein the circuitry is further configured to receive geographic location information from the client terminal via the communication interface and to preferentially acquire a subset of the second sensor data associated with a geographic region corresponding to the geographic location information.

12. The system according to claim 1, wherein the circuitry is further configured to estimate an emotion of a user by inputting multimodal sensor data into an emotion identification model, and to adjust comparison criteria used to compute the difference vector based on the estimated emotion, such that when the estimated emotion indicates stress, the circuitry applies stricter comparison criteria, and when the estimated emotion indicates relaxation, the circuitry applies relaxed comparison criteria.

13. The system according to claim 1, wherein the circuitry is further configured to retrieve, from a database, past comparison data associated with the client terminal and to select a comparison algorithm for computing the difference vector based on the past comparison data.

14. The system according to claim 1, wherein the circuitry is further configured to estimate an emotion of a user by inputting multimodal sensor data into an emotion identification model, and to adjust a display format of the response data transmitted to the client terminal based on the estimated emotion, such that when the estimated emotion indicates stress, the circuitry generates the response data in a simplified format, and when the estimated emotion indicates relaxation, the circuitry generates the response data in a detailed format.

15. The system according to claim 1, wherein the circuitry is further configured to estimate an emotion of a user by inputting multimodal sensor data into an emotion identification model, and to adjust a threshold for the anomaly score based on the estimated emotion, such that when the estimated emotion indicates stress, the circuitry applies a stricter threshold, and when the estimated emotion indicates relaxation, the circuitry applies a relaxed threshold.

16. The system according to claim 1, wherein the circuitry is further configured to retrieve, from a database, reference documents associated with the classification label using a retrieval-augmented generation architecture, and to input the reference documents together with the anomaly score into the data generation model to generate the response data.

17. The system according to claim 1, wherein the circuitry is further configured to estimate an emotion of a user by inputting multimodal sensor data into an emotion identification model, and to adjust a length of the response data based on the estimated emotion, such that when the estimated emotion indicates urgency, the circuitry generates shortened response data, and when the estimated emotion indicates relaxation, the circuitry generates lengthened response data.

18. A system comprising:
a communication interface configured to communicate with a client terminal via a packet-switched network, the client terminal comprising an image sensor, a microphone, a display, and a speaker;
a processor;
a random-access memory; and
a memory storing a data generation model obtained by deep learning on a neural network and an emotion identification model;
circuitry configured to:
receive, via the communication interface, first image tensor data captured by the image sensor of the client terminal during a first time period, the first image tensor data representing skeletal coordinate sequences extracted by a pose estimation algorithm applied to movements of a user in a reference state;
receive, via the communication interface, second image tensor data captured by the image sensor of the client terminal during a second time period subsequent to the first time period, the second image tensor data representing skeletal coordinate sequences of daily movements of the user;
extract, by inputting the first image tensor data into the neural network comprising a Transformer encoder-decoder structure with a self-attention mechanism, a first feature vector comprising joint coordinate arrays;
extract, by inputting the second image tensor data into the neural network, a second feature vector comprising joint coordinate arrays;
compute a difference vector between the first feature vector and the second feature vector in a multidimensional feature space, generate a fluctuation score based on the difference vector, and assign a fluctuation type label based on the fluctuation score;
input the fluctuation score, the fluctuation type label, and a fluctuation probability distribution into a risk evaluation neural network to generate a risk label and a risk probability;
generate, by inputting the risk label, the risk probability, and user preference information stored in a database into the data generation model comprising a large language model, response data comprising a personalized countermeasure plan; and
transmit the response data to the client terminal via the communication interface for output on the display or the speaker of the client terminal.

19. The system according to claim 18, wherein the circuitry is further configured to estimate an emotion of the user by inputting facial image data and voice waveform data received from the client terminal into the emotion identification model, and to adjust at least one of a granularity of the first image tensor data, a comparison criterion for computing the difference vector, or a format of the response data based on the estimated emotion.

20. A method performed by circuitry of a system, the method comprising:
acquiring, via a communication interface coupled to a packet-switched network, first sensor data captured by an image sensor of a client terminal during a first time period and second sensor data captured by the image sensor of the client terminal during a second time period subsequent to the first time period;
extracting, by inputting the first sensor data into a neural network comprising a Transformer encoder-decoder structure with a self-attention mechanism, a first feature vector representing a reference state;
extracting, by inputting the second sensor data into the neural network, a second feature vector representing a current state;
computing a difference vector between the first feature vector and the second feature vector in a multidimensional feature space and generating an anomaly score based on the difference vector;
generating, by inputting the anomaly score and a classification label derived from the difference vector into a data generation model, response data comprising a recommended action; and
transmitting the response data to the client terminal via the communication interface.
